(19) United States
(12) Patent Application Publication
(10) Pub. No.: US 2024/0173251 A1
(43) Pub. Date: May 30, 2024

(54) BIO-ERODIBLE OCULAR IMPLANTS FOR TREATMENT OF CONDITIONS OF THE EYE

(71) Applicant: Sight Sciences, Inc., Menlo Park, CA (US)

(72) Inventors: David Y. BADAWI, Glenview, IL (US); Paul BADAWI, Atherton, CA (US); Daniel O'KEEFFE, San Francisco, CA (US)

(21) Appl. No.: 18/522,192

(22) Filed: Nov. 28, 2023

Related U.S. Application Data
(60) Provisional application No. 63/428,389, filed on Nov. 28, 2022.

(51) Int. Cl. A61K 9/00; A61K 9/16; A61K 31/385; A61K 31/5377; A61K 31/557; A61K 38/13

(52) U.S. Cl. CPC A61K 9/0051; A61K 9/1629; A61K 31/385; A61K 31/5377; A61K 31/557; A61K 38/13

(57) ABSTRACT

The present disclosure relates to intraocular, drug-eluting microparticle implants, as well as a dry microparticle formulation comprising a plurality of drug-eluting microparticle implants, for treating a condition of the eye, wherein the implant is configured to deliver a drug to the eye without a carrier. The present disclosure also relates to methods of treating a condition of the eye by delivering one or more drugs from drug-eluting implants or a dry microparticle formulation to the anterior chamber, posterior chamber (e.g., sulcus), iridocorneal angle, sclera, cornea, limbus, subconjunctival space, sub-Tenon's space, and vitreous.

### CROSS-REFERENCE TO RELATED APPLICATION

This application claims priority to U.S. Provisional Application No. 63/428,389, filed on Nov. 28, 2022. All applications are hereby incorporated by reference in their entirety.

### TECHNICAL FIELD

This invention relates generally to bio-erodible ocular implants (e.g., drug-eluting implants) for treating conditions of the eye, and associated methods and systems for treating such conditions of the eye.

### BACKGROUND

Glaucoma is a group of optic neuropathies associated with specific structural changes to the optic nerve ultimately leading to irreversible visual field loss. In many cases, this loss of vision is progressive and leads to blindness if untreated. According to the National Eye Institute at the United States National Institutes of Health, glaucoma is the leading cause of irreversible blindness worldwide. In 2020, approximately three million people in the United States carry a diagnosis of glaucoma. Worldwide, that number is 80 million people. By 2040, it is expected that over 110 million people will be living with this potentially blinding condition (“Global Prevalence of Glaucoma and Projections of Glaucoma Burden through 2040”, Ophthalmology 2014; 121:2081-2090). Glaucoma generally falls into two categories: open angle glaucoma and closed angle glaucoma. Open angle glaucoma is approximately seven times more common than the closed angle form in both the U.S. and Europe (Quigley H A, Broman A T. Br. J. Ophthalmol. 2006; 90(3):262-267). The course of both forms of the disease is, typically, a chronic and progressive loss of vision, leading to constriction of the visual field. The ultimate result is permanent blindness. Because it is typically asymptomatic until the disease is significantly advanced, early diagnosis through regular eye exams and early treatment are critical. While the prevalence of glaucoma increases with age, the majority of patients with undiagnosed glaucoma are under 60 years of age (Shaikh Y, Yu F, Coleman A L. Am. J. Ophthalmol. 2014;158(6):1121-1129).

Risk factors associated with glaucoma include family history, ethnic origin, and age. Having a first degree relative with glaucoma is associated with a significantly increased risk (Wolfs RC, Klaver CC, Ramrattan RS, van Duijn CM, Hofman A, de Jong PT. Arch Ophthalmol. 1998; 116(12):1640-1645). Black and Hispanic individuals have increased prevalence of open angle glaucoma. Additionally, they are often diagnosed with more severe disease. Asian, Southeast Asian, Asian Indian, and Inuit individuals are more often diagnosed with closed angle glaucoma (see e.g., Varma R, Ying-Lai M, Francis B A, et al .; Los Angeles Latino Eye Study Group. Ophthalmology 2004; 111(8): 1439-1448; Tielsch J M, Sommer A, Katz J, Royall R M, Quigley H A, Javitt J. JAMA. 1991;266(3):369-374; Wormald R P, Basauri E, Wright L A, Evans J R. Eye (Lond). 1994;8(Pt 3):315-320; and Arkell S M, Lightman D A, Sommer A, Taylor H R, Korshin O M, Tielsch J M. Arch. Ophthalmol. 1987; 105(4):482-485).

Closed angle glaucoma typically results from anatomic obstruction of the anterior chamber angle and its associated drainage channels. The anatomic obstruction prevents aqueous humor from efficiently reaching the drainage channels thereby resulting in increased intraocular pressure. Surgical iridectomy, laser iridotomy, or lensectomy are often considered more definitive surgical options versus more palliative medical therapy such as cholinergic drugs (e.g., pilocarpine eyedrops) to relive obstruction by pupil constriction.

Open angle glaucoma (OAG) is much more common in the U.S., and it accounts for significantly more loss of vision that its closed counterpart. While the exact pathophysiology of OAG is not completely understood, it has been demonstrated that increased intraocular pressure (IOP) correlates with retinal ganglion cell death. There is a relationship between secretion of aqueous humor by the ciliary body and its egress from the eye via conventional trabecular meshwork pathways and the unconventional uveoscleral pathway. This relationship and any resultant imbalances determine IOP. It is felt that an increased resistance to outflow in the trabecular meshwork or more distal aqueous collector channels are associated with increased IOP in OAG. Increased IOP may cause mechanical stress on the lamina cribrosa, where retinal ganglion cell axons exit the eye to coalesce into the optic nerve. IOP-induced stress at the lamina cribrosa can deform, damage, and interfere with the retinal axons leading to irreversible injury and vision loss. While such IOP associated damage typically occurs when the pressure is above the population average pressures, it can occur at lower or “normal” pressure depending on an individual's vulnerability. Conversely, many people with higher-than-average IOP never develop glaucoma. A growing number of studies are identifying genomic loci associated with glaucoma susceptibility. Thus, glaucoma may develop in patients with an intraocular pressure relatively high for their individual susceptibility (see e.g., Thorleifsson G, Walters G B, Hewitt A W, et al. Nat Genet. 2010;42(10):906-909; and Wiggs J L, Yaspan B L, Hauser M A, et al. PLOS Genet. 2012;8(4):e1002654). When ganglion cell death does occur in glaucoma, characteristic changes in the optic nerve head and the nerve fiber layer become evident. This eventually is associated with characteristic visual field loss patterns. Prompt referral to an eye care specialist is critical to treat the glaucoma and slow the progression of irreversible damage and subsequent loss of vision. There is no single gold standard test for diagnosing glaucoma. Typically, several criteria are taken into consideration in making the diagnosis of glaucoma. These include age, family history, ethnic background, IOP, corneal thickness, optical coherence tomographic analysis of various retinal tissues, optic nerve head appearance, and peripheral visual field testing.

The primary goal of treatment is to slow progressive optic nerve damage in order to preserve vision and quality of life. Early diagnosis and intervention are critical given that visual loss is irreversible. Reduction of IOP with treatment combined with continuous diagnostic assessments of treatment efficacy are part of the mainstay of glaucoma care.

Initially, treatment is typically comprised of the least number of medications required to adequately reduce IOP. Medications include drugs from the following families of compounds: prostaglandins, prostaglandin analogs, beta-adrenergic blockers, alpha-adrenergic agonists, carbonic anhydrase inhibitors, Rho-kinase (ROCK) inhibitors, and cholinergic drugs.

Should medical therapy fail, not be tolerated, or not be possible, other forms of therapy may be added or substitute to medical therapy. For example, laser therapy to the eye in the form of trabeculoplasty, cycloablation (endoscopic or transscleral) may be performed. In more advanced cases or under some circumstances, incisional surgery can be considered. Trabeculectomy, valves, or shunts can be used to help control IOP. Recently, minimally invasive glaucoma surgery or MIGS has become a popular surgical approach to the treatment of glaucoma. Various technologies are being employed to reduce IOP while reducing exposure to surgical risks posed by more invasive treatments like trabeculectomy or valve placement. In 2017, nearly 175,000 surgical procedures were performed. The surgeries included over 20,000 trabeculectomies, 20,000 glaucoma drainage implants, and over 130,000 MIGS procedures (Ma A K, Lee J H, Warren J L, Teng C C. Clin Ophthalmol. 2020; 14:2551-2560).

While medical therapy is the preferred initial treatment of OAG in the U.S., it does have many problems. Drops can be cost prohibitive for patients, and patients can forget to regularly use them. Additionally, proper instillation into the conjunctival cul-de-sac may be more difficult, especially in the hands of the elderly or arthritic. Excessive instillation, such as instilling multiple drops, and subsequent wasting of medication is also an issue. However, even with proper instillation of the eyedrops, medication is wasted. For instance, a typical eyedrop may be 60-90 microliters, but the ocular surface can typically hold no more than 10 microliters. The therapeutic ingredients and the preservatives with which they are often combined can lead to ocular surface disease, discomfort, inflammation, dry eye, and reduced corneal sensitivity, all of which can irritate the eye and further reduce compliance. Multiple eyedrop medications can also lead to confusion and misuse of the medications. All of these factors combine to create problems with the mainstay of glaucoma therapy-drugs. However, medications do avoid a lot of the more serious complications that can occur with surgery.

Surgical therapy of glaucoma is typically reserved as a second line therapy in the U.S. That is gradually changing with microinvasive glaucoma surgery (MIGS) becoming more mainstream. Nonetheless, glaucoma surgery carries its own risks. One of the more problematic complications is bacterial endophthalmitis, which is a potentially visually devastating ocular infection. However, there are many other complications of glaucoma surgery, including failure, hypotony, hemorrhage, malignant glaucoma, progression, hyphema, retinal detachment, and many others. Moreover, there are long term complications with trabeculectomy plus antimetabolite and glaucoma drainage devices.

While glaucoma has been covered in more detail here, many other ocular diseases are being successfully treated with drugs. In addition to being used to medically treat various ocular diseases, drugs are also often used as an adjunct in the surgical treatment of ocular diseases. They are used to treat ocular surface disease, corneal disease, scleral disease, uveal disease, vitreous disease, and chorioretinal disease.

Age-related macular degeneration (AMD) is a common cause of vision loss in the U.S., and it is globally the third leading cause of blindness. It is associated with degeneration of the retinal pigment epithelium and Bruch's membrane. This itself can lead to overlying retinal damage and visual loss. Unlike the aforementioned “dry” degeneration, further degeneration and resulting in growth of neovascularization from the underlying choriocapillaris can lead to significant visual loss. This latter process is called “wet” macular degeneration. One of the more common forms of treatment is regular intravitreal injections of antibodies or drugs targeting vascular endothelial-derived growth factor (VEGF). Patients often need to receive such injections with anti-VEGF drugs, typically antibody-derived therapeutics, including ranibizumab, aflibercept, or bevacizumab every 4-8 weeks to control their wet macular degeneration. There are significant issues with cost, complications, and associated morbidity with this mainstay of treatment.

Other retinal diseases require drug treatment, including macular edema, vascular occlusions, diabetic retinopathy, retinal degenerations, and retinal dystrophies. Uveal diseases can affect the choroid, ciliary body, and iris. Examples include iritis and other forms of uveitis which can respond well to various drugs such as steroids. Steroids are often given as oral treatments or as topical therapy. More aggressive cytotoxic agents and chemotherapy can be employed for more severe cases like Behcet's disease.

The vitreous can also be a location for ocular disease. It may harbor vision obstructing opacities or hemorrhage. In other instances, it may accumulate inflammatory cells in the setting of vitritis which can also lead to visual loss.

The lens in the eye is subject to a number of diseases. The most common are age related. Lens opacity or cataract often requires surgical correction in the form of cataract surgery. While many drugs have been used to slow cataract formation, none have so far proven to be significantly effective. After cataract surgery, a variety of drugs are often used to reduce likelihood of infection or inflammation.

The cornea can become opaque, scarred, or deformed because of diseases such as herpes zoster or simplex infection, keratitis, keratoconus, various infectious diseases, or other corneal degenerations. Additionally, transplanted corneas can suffer from immune mediated rejection or recurrence of a primary ocular disease.

Scleral disease can result from immune processes or infections. In fact, one of the most common ocular diseases is myopia or nearsightedness, and it is believed that the sclera plays a critical role in axial length and refractive state of the eye. Dilute topical atropine which may work by inducing cycloplegia, or paralysis of the ciliary body, has been shown to reduce development of axial myopia in children. Complex pathways likely account for the development of myopia in children. There is great interest in this area given the large percentage of the world's population that is affected.

Dry eye disease (DED) encompasses a number of disease states, defined by the TFOS DEWS II (Ocul. Surf., 2017, 15(3), 269-650) as “a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles.” DED can be generally divided into two classes: aqueous tear-deficient dry eye (ADDE) and evaporative dry eye (EDE), each of which can be divided further into subclasses. One of the leading conditions associated with DED, and in particular EDE, is meibomian gland dysfunction (MGD), which is itself an umbrella term encompassing several disorders, wherein disruption and obstruction of the meibomian gland negatively impacts the quality and quantity of meibum, a lipid-rich secretion that protects the ocular surface from damage and premature evaporation of tears (Chhadva, et al., Ophthalmology, 2017, 124(11 Suppl), S20-S26). There are approximately 20-40 meibomian glands in each eyelid and they are responsible for producing meibum that coats the tears and prevents premature tear evaporation. When the meibomian glands are healthy, patent, and functioning properly, meibum assumes a liquid, olive-oil like consistency. Every blink applies an expression force to the meibomian glands and some clear liquid meibum is expressed from the gland orifice and fortifies the tear's outermost lipid layer. With MGD-based EDE, the development of an imbalance in natural lipids and lipid chemistry results in a higher meibum melting temperature, which ultimately leads to meibum transitioning from a healthy, clear, liquid state to a cloudy, semi-hardened state to an advanced, diseased, hardened state. Eventually as the disease progresses and the lipid chemistry of meibum worsens, the hardened meibum becomes unavailable and inexpressible resulting in a low-quality tear lipid layer and premature tear evaporation. In obstructive MGD, the blink results in little to no expression of meibum due to its hardened physiochemical state and inexpressibility, a compromised lipid layer, and accelerated tear evaporation.

Globally, the prevalence of dry eye disease (DED) is estimated to be between 5 to 20 percent, and approximately 16 million Americans have been diagnosed. It is estimated that 86% of these DED sufferers have MGD-associated EDE (Lemp, et al., Cornea, 2012, 472-478). Those with DED suffer from either inadequate tear production, poor quality of tears, or both, which results in redness, stinging, burning, itching, light sensitivity, watery eyes, blurry vision, irregularities of the ocular surface, and damage to corneal or conjunctival epithelium and tissues. It is believed that a majority of dry eye patients have at least some degree of both aqueous deficiency and lipid deficiency. Treatment is largely palliative, and no broad cure for DED has been developed.

Drugs can be administered for these, and other, ocular diseases in various ways. While there are systemic routes of administration such as oral or intravenous drug administration, the eye is particularly well suited to local administration because of its location on the surface of the body. Thus, local routes of drug administration are preferred in the majority of cases. This allows limited exposure of the rest of the body to drug and reduces the amount of drug needed. Currently, the most common route of drug administration for glaucoma is a topical eyedrop approach. Such topically administered drugs typically diffuse across the cornea and into the eye. For retinal diseases, drug injection is a common route.

Given the above-described difficulty in treating diseases such as, for example, glaucoma, age-related macular degeneration, and dry eye disease, there is a need for safer, more effective, and convenient treatments which address the shortcomings of the current standards of care.

### SUMMARY

Drug-eluting implants for treating a condition of the eye, and methods for their use, are disclosed herein. In some variations, a drug-eluting implant is a bio-erodible microsphere. Such implants are configured to be implanted in an eye of a subject without a liquid carrier, for example in the form of a dry implant formulation comprising a plurality of implants.

Also disclosed herein are methods for treating a condition of an eye of a subject. These methods may comprise implanting at least one drug-eluting implant in the eye of the subject without using a liquid carrier. In such methods, a drug may be delivered from at least one drug-eluting implant to reduce a symptom of a condition of an eye.

### BRIEF DESCRIPTION OF THE DRAWINGS

FIG. 1 shows a cross-sectional view of the anatomy of a normal human eye.

FIG. 2 shows exemplary drug-eluting implants.

FIGS. 3A-3D shows an exemplary implantation system.

FIG. 3E shows an alternative drive assembly comprised in an implantation system.

FIG. 4 shows a flowchart of a method for loading a dry implant formulation into an embodiment of a cannula of an implantation system.

FIGS. 5A-5B show an exemplary method of dry implant formulation into an embodiment of a cannula of an implantation system.

FIG. 6 shows a flowchart of a method for implanting a dry implant formulation.

FIG. 7 shows an exemplary method of implanting a dry implant formulation in the eye to treat an ocular condition.

### DETAILED DESCRIPTION

### Overview

Described herein are devices, systems, and methods for treating conditions of the eye (e.g., glaucoma, dry eye disease, and others as described herein). Generally, such devices are intended to be implanted in the eye (e.g., within one or more of the sulcus, posterior chamber, anterior chamber, vitreous, suprachoroidal space, subretinal space, retrobulbar space, peribulbar space, intracapsular space, Tenon's capsule, sub-Tenon's space, intrascleral space, subconjunctival space, intra-lenticular capsule space, Berger's space) to release one or more drugs to one or more regions impacted by a disease or condition of the eye. For instance, the devices described herein may be intraocular implants.

The devices described herein may generally comprise intraocular, drug-eluting implants for treating one or more conditions of the eye. The drug-eluting implants may comprise a drug-eluting matrix configured to release one or more drugs into the eye. The implants and/or drug-eluting matrices of the implants may comprise a bio-erodible material (e.g., a bio-erodible polymer) that degrades over a predetermined period of days, weeks, months, or years, delivering the drug or drugs over a portion or the entirety of that same time period. Additionally or alternatively, the drug-eluting implants and/or drug-eluting matrices of the implants may comprise a material that preferentially release the drug or drugs when in an aqueous environment, such as in tear, humor, or serum, for example when in the eye or a portion thereof, as compared to a dry environment. In some variations, the drug-eluting implants may be spherical (e.g., microspheres), spheroidal, ellipsoid, or ovoid. The drug-eluting implants may be roughly spherical. In some variations, the drug-eluting implants may be a needle- or rod-shaped. In some variations, the drug-eluting implants may be a cuboid. In some variations, the drug-eluting implants may be a pellet or a particle. In some variations, the drug-eluting implants may be irregular in shape. The drug-eluting implants may be a sub-millimeter (“sub-mm”) drug-eluting implant, which is dimensioned so that the maximum linear dimension of the implant (e.g. diameter if spherical, major axis if ovoid, space diagonal if cuboid) is less than a millimeter. As used herein, a sub-mm, drug-eluting implant may be referred to as a “microparticle implant”. The drug-eluting implants may be a sub-micron drug-eluting implant, which is dimensioned so that the maximum linear dimension is less than a micron. As used herein, a sub-micron drug-eluting implant may be referred to as a “nanoparticle implant”.

Also described herein are implant formulations that may comprise a plurality of microparticle implants as well as implantation systems configured to deliver an implant formulation to an eye of a subject. In some variations, the implant formulation may be a dry implant formulation without a liquid carrier, and the implantation system may be configured to deliver to an eye of a subject the dry implant formulation without a liquid carrier, In some variations, one or more microparticle implants, optionally in the form of an implant formulation (e.g. a dry implant formulation as described herein), and one or more implantation devices or systems (e.g., two, three, four, or more) may be packaged as a kit. In some instances, one or more drug-eluting implants or an implant formulation (e.g. a dry implant formulation as described herein), may be preloaded in a corresponding implantation system, while in other variations, one or more implants may be provided separately from an implantation system, and the one or more implants may be loaded into or otherwise positioned within an implantation system (e.g., within a cannula of an implantation system) by a user. In some of these variations, the one or more implants may be provided separately and may be prepared as an implant formulation (e.g. a dry implant formulation), and the implant formulation may be loaded into or otherwise positioned within an implantation system (e.g., within a cannula of an implantation system). In some variations, multiple implants, or multiple implant formulations, may be delivered sequentially. In some variations, multiple implants, or multiple implant formulations, may be delivered simultaneously. For instance, 2, 3, 4, 5, 6, 7, 8, 9, 10, 50, 100 or more implants or implant formulations may be delivered sequentially or simultaneously.

Methods for treating conditions of the eye may generally comprise advancing one or more drug-eluting implants (e.g., microspheres), or an implant formulation comprising a plurality of implants (e.g., a dry implant formulation as disclosed herein), to a target location (e.g., an implantation site) in the eye, positioning the one or more drug-eluting implants or implant formulations in one or more target locations in the eye, and delivering one or more drugs from the implant(s) to the target location(s), and/or to other locations in the eye to treat the condition of the eye and/or reduce one or more symptoms associated with the condition of the eye. For example, in some variations, the target location may be the subconjunctival space, and the methods may generally comprise advancing one or more drug-eluting implants or dry implant formulations through the conjunctiva, positioning the one or more implants or implant formulations (e.g., a dry implant formulation as disclosed herein), in the subconjunctival space, and delivering one or more drugs from the implant(s) to the subconjunctival space to treat the condition of the eye and/or to reduce one or more symptoms of the condition of the eye. In some variations, the target location may be the anterior chamber, and the methods may generally comprise advancing a drug-eluting implant or an implant formulation (e.g., a dry implant formulation as disclosed herein) through the conjunctiva and the cornea of the eye, positioning the implant or implant formulation in the anterior chamber, and delivering a drug from the implant to the anterior chamber to treat the condition of the eye and/or to reduce one or more symptoms of the condition of the eye. In some variations, the target location may be the vitreous, and the methods may generally comprise advancing and positioning a drug-eluting implant or implant formulation (e.g., a dry implant formulation as disclosed herein) into the vitreous to treat the condition of the eye and/or to reduce one or more symptoms of the condition of the eye. In some variations, the target location may be the suprachoroidal space, and the methods may generally comprise advancing and positioning a drug-eluting implant or implant formulation (e.g., a dry implant formulation as disclosed herein) into the suprachoroidal space, optionally via an ab externo approach or ab interno approach through the anterior chamber angle, to treat the condition of the eye and/or to reduce one or more symptoms of the condition of the eye. Additionally or alternatively, such implants or implant formulations (e.g., a dry implant formulation as disclosed herein) may be advanced using an implantation system. Conditions of the eye may include, but are not limited to, ocular surface diseases, corneal diseases, scleral diseases, uveal diseases, vitreous diseases, optic nerve diseases, choroidal diseases, and retinal diseases. Implantation of the drug-eluting implants or implant formulations (e.g., a dry implant formulation as disclosed herein) may be performed in a doctor's office by general ophthalmologists and thus do not require costly surgery performed by a specialist. Additionally or alternatively, implantation of the drug-eluting implants or implant formulations (e.g., a dry implant formulation as disclosed herein) may be done in an operating room as a standalone procedure or combined with other procedures (e.g., cataract surgery).

Implants or implant formulations (e.g., a dry implant formulation as disclosed herein) that reside in the posterior chamber (e.g., within the sulcus, within the sulcus and partially extending into the remainder of the posterior chamber) may utilize naturally occurring currents (e.g., anterior flowing currents, posteriorly flowing currents), that may deliver one or more drugs to different portions of the eye. For example, it has been shown that aqueous humor produced at the ciliary body and released into the ciliary sulcus and/or posterior chamber can travel anteriorly into the anterior chamber where it travels via convection currents. Additionally, posterior aqueous currents may reach the retina and thus deliver drugs to the retina and choroid (e.g. anti-vascular endothelial growth factor antibodies for exudative macular degeneration).

Accordingly, the devices, systems, and methods described herein may provide effective treatment for many conditions of the eye while avoiding the costs and consequences of utilizing formal operating rooms, decreasing the total amount of drug needed, reducing or eliminating systemic drug exposure through spatially targeted implantation, decreasing or eliminating the need for frequent injections or daily eyedrops, and increasing patient compliance with suggested treatment regimens by eliminating repeated administration of eye drops or injections used in conventional treatments. The devices, systems, and methods described herein may deliver a much smaller amount of drug to target tissue without compromising on efficacy, and thus drug and preservative side effects may be reduced. For example, whereas timolol is potentially effective in treating elevated ocular pressure, systemically available timolol is known in the art to cause cardiac and pulmonary complications. This safety issue imposes a limitation on the dosage and frequency of administration when using timolol eye drops for treating elevated ocular pressure. Dry delivery of timolol-eluting implants in the eye (e.g. in the subconjunctival space or sub-Tenon's space) would make it possible to deliver a timolol, when compared to eye drops, at a higher concentration in the eye and a lower concentration systemically (e.g., in serum). Also for example, dorzolamide hydrochloride eye drops typically cause pain and discomfort due to the low pH that is typically required to dissolve the dorzolamide into solution. Dry delivery of dorzolamide hydrochloride, dorzolamide base, or brinzolamide-eluting implants would eliminate pH or solubility issues seen with topical administration, and reduce the pain and/or discomfort associated with dorzolamide or brinzolamide eye drops.

### Anatomy

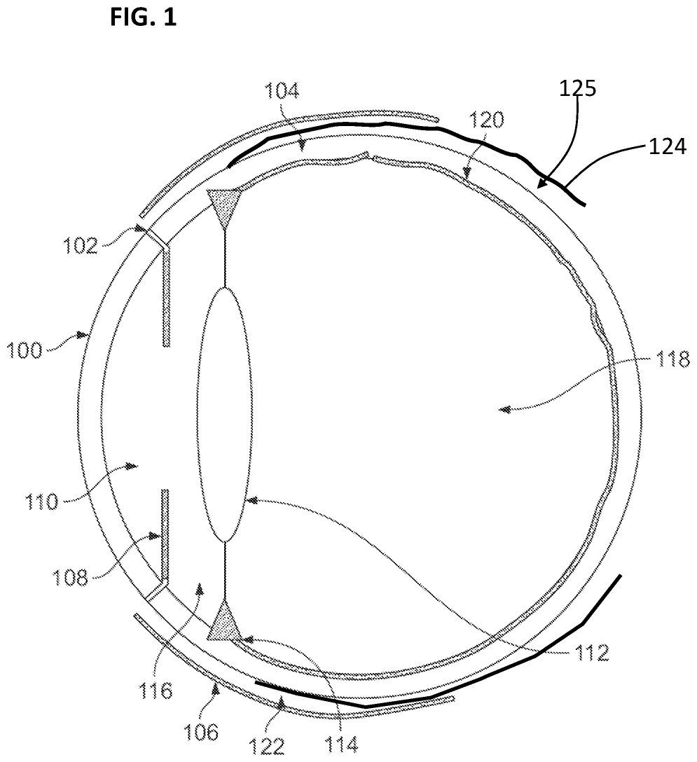

For context, FIG. 1 shows a partial cross-sectional view of the anatomy of a normal human eye. The eye can be conceptualized as a fluid filled sphere. Anteriorly, it is bounded by the cornea (100), a three-layered clear tissue that allows entry of light, and functions like a protective window allowing for entry of light into the eye. The periphery of the cornea (100) is known as the corneal limbus (“limbus”) (102) which defines the junction with the sclera (104). The limbus (102) contains stem cells for the ocular surface, contains numerous aqueous outflow pathways, and is highly vascularized.

The sclera (104) is the opaque, tough, protective, outer layer of the eye. Like the cornea it is essentially avascular. Overlying the sclera (104) is the conjunctiva (106), the thin, clear tissue that overlies the sclera (104) and the inside of the eyelids. By contributing mucus and tears, it helps lubricate the ocular surface. In addition, it is vascularized, and it helps contribute to ocular immune responses. The space below the conjunctiva is the subconjunctival space (122). Tenon's capsule (124) is a membrane that covers the outside of the eyeball between the conjunctiva and the sclera. Tenon's capsule contributes to the structural integrity of the eye, and provides another layer of protection for the eyeball. Sub-Tenon's space (125), which may also be referred to as episcleral space, is a space between the Tenon's capsule and the sclera. Sub-Tenon's space contains loose connective tissue, blood vessels, and fat, and allows for smooth movements of the eyeball by providing a cushioning effect.

Posterior to the cornea, is the iris (108), or the colored part of the eye. It is an annular structure which can adjust its aperture (pupil) to regulate the amount of light entering the eye. Bright light causes constriction of the pupil thereby limiting exposure to excessive light or resulting glare. Under dim lighting, the pupil opens to capture more of the available light.

The anterior chamber angle (110), which is filled with aqueous humor, resides between the iris and cornea. At its periphery, there is the anterior chamber angle (110), where aqueous drains out of the eye through the trabecular meshwork and Schlemm's canal. A circular band of the ciliary body is seen on gonioscopy. This area provides intracameral access to the suprachoroidal space.

Behind the iris (108) is the lens (112). The normal lens is transparent, and it focuses light on the retina to create a clear image. With age or disease, the lens (112) may cloud, and this is known as cataract. The lens (112) is suspended in the eye by fibers known as lens zonules. One end of the zonules attach around the equator of the lens (112). The other end of the zonules attaches to the ciliary body (114). Contraction and relaxation of the ciliary body alter load on the zonules thereby resulting in increased curving of the lens or flattening of the lens (112). This is the primary mechanism our eyes use for focusing.

The ciliary body (114) not only contains muscles that apply load to the zonules, but it is also responsible for secreting aqueous humor which travels through the ciliary sulcus (116), the peripheral part of the posterior chamber. Implants or devices residing in the ciliary sulcus or peripheral posterior chamber avoid the visual axis and thus do not interfere with vision. Aqueous humor flows into the ciliary sulcus and posterior chamber into the pupil and ultimately into the anterior chamber (110). Research has also shown that aqueous humor currents can also drive fluid and substances through the vitreous and through the retina. In other words, the currents are bidirectional. The posterior chamber is the space in the eye behind the iris and in front of the lens. At its periphery, it is bounded by the ciliary sulcus (116). The ciliary sulcus (116) is the space between the front of the ciliary body (114) and the posterior surface of the iris. This part of the posterior chamber is typically 12 mm in diameter.

The vitreous humor (118) is the gelatinous substance filling the central cavity of the eye. Its volume is approximately 4-4.5 mL (milliliters). It is bounded by the retina peripherally and posteriorly. Anteriorly, it is bounded by Berger's space, which separates the vitreous cavity from the lens centrally and by the canal of Petit, also known as spatia zonularis, which separates it from the lens peripherally. The retina is the photosensitive nerve layer lining the back of the eye. In humans, the retina has ten layers, with the outermost, or closest to the sclera (104), being the retinal pigment epithelium. This layer has been implicated in macular degeneration.

Between the sclera and the retina is a part of the uvea known as the choroid (120). It is a high flow, low resistance vascular layer that nourishes and oxygenates the outer two thirds of the retina. It has also been implicated in macular degeneration. The macula is a region of the retina that accounts for high contrast, crisp vision. It is the functional center of the retina and gives humans their central vision. For example, the ability to read or recognize faces clearly is dependent on the macula. Macular degeneration affects this area, and thus can have devastating impact on vision. The optic nerve is the coalescence of approximately 1 million retinal axons carrying visual information from the eye to vision centers in the brain.

### Drug-Eluting Implants

In general, the devices, systems, and formulations described herein comprise drug-eluting implants for treating one or more conditions of the eye by delivering one or more drugs to the eye. The implants may comprise a round shape (e.g., spherical, generally spherical, spheroidal, ovoid, ellipsoid), and/or an irregular shape, and may be configured to be implanted within one or more structures and/or cavities of the eye. For instance, the drug-eluting implant(s) may be configured to reside partially or entirely within one or more of the subconjunctival space, the anterior chamber, the sulcus, the posterior chamber, the suprachoroidal space, the sub-retinal space, or the vitreous of one or both eyes. In some variations, as will be discussed in more detail herein, the implants may be microparticle implants, e.g., microspheres.

In some variations, as will be discussed in more detail herein, the implants may be configured to be delivered without a carrier (e.g., vehicles and the like). As used herein, a carrier is defined as a substance (e.g., a fluid) used as a medium for administration of a pharmaceutical compound or pharmaceutical device. For instance, an implant could be delivered without a carrier (i.e., dry), or it could be delivered with a carrier (e.g., one or more implants suspended in a fluid). It will be appreciated that delivering implants with non-fluid additives or compositions for example binding agents (e.g. sucrose, gelatin or cellulose) would be considered dry delivery in accordance with the disclosure.

The implants may comprise one or more drugs, and may be configured to release or elute the one or more drugs over time (e.g., a predetermined period of time). In certain variations, the implants may be configured or formulated to preferentially release the one or more drugs when in an aqueous environment, such as in tear, humor, interstitial fluid, or serum, for example when in the eye or a portion thereof, as compared to a dry environment. In variations in which the drug-eluting implant is a microparticle implant (e.g., a microsphere), the microparticle implant may comprise the one or more drugs, such as within a drug-eluting matrix forming the microparticle implant and/or in a coating on an exterior surface thereof. Regardless, the implants may be drug-eluting or otherwise configured to deliver, administer, or provide one or more drugs to the eye.

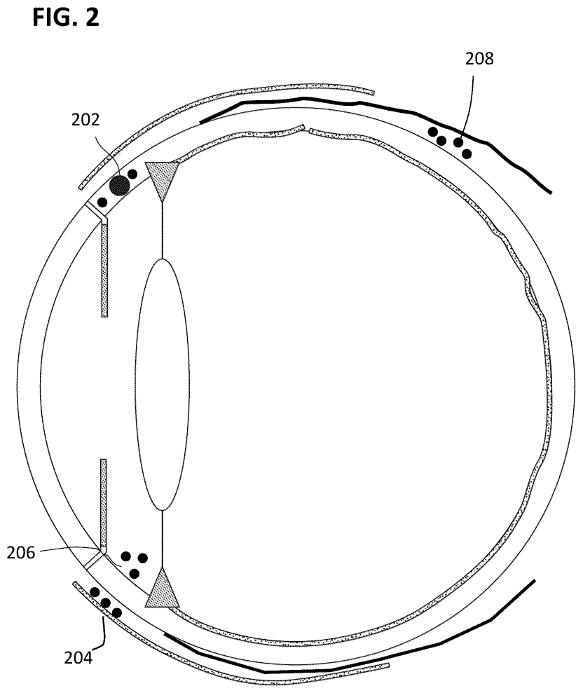

FIG. 2 depicts exemplary drug-eluting implants described herein, where a plurality of the drug-eluting implants (202) reside intramurally in the sclera, where a plurality of the drug-eluting implants (204) reside within the subconjunctival space, where a plurality of the drug-eluting implants (206) reside within the sulcus, and where a plurality of the drug-eluting implants (208) reside within the sub-Tenon's space.

As shown in FIG. 2, the drug-eluting implant may comprise a rounded shape, and may be, for example, a microsphere, a microspheroid, a microovoid, a microellipsoid, or the like. In some variations, Thus, the round drug-eluting implants described herein may have a variety of cross-sectional shapes, including, for example, circular, oval, elliptical, or the like. In some variations, the drug-eluting implant may be any other shape as discussed above, e.g., needle shaped, rod-shaped, cuboid, or an irregular shape. In general, the implant, or a portion of the implant, may be solid. In some variations, all or a portion of the implant may be a drug-eluting matrix. For example, in some variations, the entire external surface of the implant may be coated with the drug-eluting matrix or only a portion (e.g., one third, one half, two-thirds, or the like) of the implant may be coated. In some variations, the drug-eluting implant may consist essentially of the drug-eluting matrix. In some variations, the drug-eluting implant may comprise the drug-eluting matrix and one or more additional substances that control elution rate, control degradation rate or provide biocompatibility. The one or more additional substances may be mixed with the drug-eluting matrix, or coat a surface of the drug-eluting implant. In some variations, the drug-eluting matrix may comprise a mixture, optionally an evenly dispersed mixture, of one or more drugs and one or more bio-erodible polymers.

In some variations, the microparticle implants described herein may be characterized by a maximum linear dimension (a diameter if spherical, a major axis if ovoid,). The maximum linear dimension may be between about 0.1 μm and about 500 μm. In some variations, the implants may, independently, have a maximum linear dimension of between about 0.1 μm and about 40 μm, between about 0.1 μm and about 30 μm, between about 0.1 μm and about 20 μm, between about 0.1 μm and about 10 μm, between about 0.1 μm and about 1 μm, between about 0.1 μm and about 400 μm, between about 0.1 μm and about 300 μm, between about 0.1 μm and about 200 μm, between about 0.1 μm and about 100 μm, between about 0.1 μm and about 10 μm, between about 0.5 μm and about 40 μm, between about 0.5 μm and about 30 μm, between about 0.5 μm and about 20 μm, between about 0.5 μm and about 10 μm, between about 1 μm and about 400 μm, between about 1 μm and about 300 μm, between about 1 μm and about 200 μm, between about 1 μm and about 100 μm, between about 1 μm and about 10 μm, between about 1 μm and about 5 μm, between about 5 μm and about 100 μm, between about 10 μm and about 100 μm, between about 10 μm and about 90 μm, between about 10 μm and about 80 μm, between about 10 μm and about 70 μm, between about 10 μm and about 60 μm, between about 10 μm and about 50 μm, between about 10 μm and about 40 μm, between about 10 μm and about 30 μm, between about 10 μm and about 20 μm, between about 20 μm and about 100 μm, between about 20 μm and about 90 μm, between about 20 μm and about 80 μm, between about 20 μm and about 70 μm, between about 20 μm and about 60 μm, between about 20 μm and about 50 μm, between about 20 μm and about 40 μm, between about 20 μm and about 30 μm, between about 40 μm and about 100 μm, between about 30 μm and about 90 μm, between about 30 μm and about 80 μm, between about 30 μm and about 70 μm, between about 30 μm and about 60 μm, between about 30 μm and about 50 μm, between about 30 μm and about 40 μm, between about 40 μm and about 100 μm, between about 40 μm and about 90 μm, between about 40 μm and about 80 μm, between about 40 μm and about 70 μm, between about 40 μm and about 60 μm, between about 40 μm and about 50 μm, between about 50 μm and about 100 μm, between about 50 μm and about 90 μm, between about 50 μm and about 80 μm, between about 50 μm and about 70 μm, between about 50 μm and about 60 μm, between about 60 μm and about 100 μm, between about 60 μm and about 90 μm, between about 60 μm and about 80 μm, between about 60 μm and about 70 μm, between about 70 μm and about 100 μm, between about 70 μm and about 90 μm, between about 70 μm and about 80 μm, between about 80 μm and about 100 μm, between about 80 μm and about 90 um; or between about 90 μm and about 100 μm, (including all sub-ranges and values therein). In some variations, an implant may have a maximum linear dimension of about 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 150, 200, 250, 300, 350, 400, 450, or 500 μm.

In some variations, a mixture of implants (e.g., different sized implants, different shaped implants, implants with different drugs) described herein may be delivered. In some instances, it may be beneficial to administer a mixture of the implants described herein, in which the mixture contains implants of a similar size (e.g., a majority of the implants have a maximum linear dimension, which may be a diameter in the case of microsphere implants, within 5%, 10%, 15%, or 20% of the mean maximum linear dimension of the implants in the mixture). Without being bound by theory, using implants of a similar size, for example, in a dry implant formulation, may aid in preventing formation of a lattice between individual implants, and this prevention of lattice formation may reduce or prevent clogging of an implantation system loaded with a plurality of implants or a dry implant formulation.

When two or more implants are delivered (for example as an implant formulation), they may collectively have a mean maximum linear dimension. For instance, in some variations, two or more implants may have a mean maximum linear dimension of between about 0.1 μm and about 500 μm. In some variations, the implants may, collectively, have a mean maximum linear dimension of between about 0.1 μm and about 40 μm, between about 0.1 μm and about 30 μm, between about 0.1 μm and about 20 μm, between about 0.1 μm and about 10 μm, between about 0.1 μm and about 1 μm, between about 0.1 μm and about 400 μm, between about 0.1 μm and about 300 μm, between about 0.1 μm and about 200 μm, between about 0.1 μm and about 100 μm, between about 0.1 μm and about 10 μm, between about 0.5 μm and about 40 μm, between about 0.5 μm and about 30 μm, between about 0.5 μm and about 20 μm, between about 0.5 μm and about 10 μm, between about 1 μm and about 400 μm, between about 1 μm and about 300 μm, between about 1 μm and about 200 μm, between about 1 μm and about 100 μm, between about 1 μm and about 10 μm, between about 1 μm and about 5 μm, between about 5 μm and about 100 μm, between about 10 μm and about 100 μm, between about 10 μm and about 90 μm, between about 10 μm and about 80 μm, between about 10 μm and about 70 μm, between about 10 μm and about 60 μm, between about 10 μm and about 50 μm, between about 10 μm and about 40 μm, between about 10 μm and about 30 μm, between about 10 μm and about 20 μm, between about 20 μm and about 100 μm, between about 20 μm and about 90 μm, between about 20 μm and about 80 μm, between about 20 μm and about 70 μm, between about 20 μm and about 60 μm, between about 20 μm and about 50 μm, between about 20 μm and about 40 μm, between about 20 μm and about 30 μm, between about 40 μm and about 100 μm, between about 30 μm and about 90 μm, between about 30 μm and about 80 μm, between about 30 μm and about 70 μm, between about 30 μm and about 60 μm, between about 30 μm and about 50 μm, between about 30 μm and about 40 μm, between about 40 μm and about 100 μm, between about 40 μm and about 90 μm, between about 40 μm and about 80 μm, between about 40 μm and about 70 μm, between about 40 μm and about 60 μm, between about 40 μm and about 50 μm, between about 50 μm and about 100 μm, between about 50 μm and about 90 μm, between about 50 μm and about 80 μm, between about 50 μm and about 70 μm, between about 50 μm and about 60 μm, between about 60 μm and about 100 μm, between about 60 μm and about 90 μm, between about 60 μm and about 80 μm, between about 60 μm and about 70 μm, between about 70 μm and about 100 μm, between about 70 μm and about 90 μm, between about 70 μm and about 80 μm, between about 80 μm and about 100 μm, between about 80 μm and about 90 μm; or between about 90 μm and about 100 μm, (including all sub-ranges and values therein). In some variations, implants may have, collectively, an average maximum linear dimension of about 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 150, 200, 250, 300, 350, 400, 450, or 500 μm.

The implants (e.g., microparticles such as microspheres) described herein may comprise a drug-eluting matrix. As used herein, a “drug-eluting matrix” refers to a material impregnated with a drug, where the drug is released from the material when positioned within the eye. In some variations, the drug may be released slowly from the material over a set period of time. The drug-eluting matrix may, for instance, comprise a polymer impregnated with the drug, where the drug is released from the polymer while implanted within the eye. In certain embodiments, the drug-eluting matrix may be erodible such that it dissolves or disintegrates within a predetermined period of time inside the subject, safely, into non-toxic or biocompatible components. In some embodiments, the entire microsphere is comprised of the drug-eluting matrix. The drug-eluting matrices described herein may form a coating on, or a filler within, a structure of the drug-eluting implants described herein. For instance, in some embodiments, the microsphere may comprise a hollow interior chamber containing the drug-eluting matrix and the microsphere may further comprise fenestrations (e.g., openings) through a wall of the microsphere to deliver the drug from the drug-eluting matrix in the interior chamber to the eye. Additionally or alternatively, all or a portion of the implant may comprise a drug-eluting matrix in the form of a coating.

The implants (e.g., microparticles such as microspheres) described herein may comprise, in part or in whole, a variety of materials suitable for use in a human subject, such as one or more biocompatible polymers or plastics or polymer composites. Examples of biocompatible polymers include poly(lactic-co-glycolic acid) (PLGA), poly(lactic acid) (PLA), poly(glycolic acid) (PGA), poly-epsilon-caprolactone (PCL), high density polyethylene (HDPE), poly(styrene-block-isobutylene-block-styrene) (SIBS), polyurethane, polycarbonate, polypropylene, polymethylmethacrylate (PMMA), polybutylmethacrylate, polyesters, polytetrafluoroethylene (PTFE), silicone, acrylic polymers, polyvinyl alcohol, polyvinyl pyrrolidone, polyvinyl chloride, ethyl vinyl acetate, collagen, collagen derivatives, flexible fused silica, polyolefins, NYLON® polymers, polyimide, polyacrylamide, fluorinated elastomers, and copolymers and blends thereof. In some variations, the biocompatible polymer may be a thermoresponsive polymer, such as Poly(N-isopropylacrylamide) (PNIPAM). The implant (e.g., microsphere) may be fully or partially bio-erodible (e.g., biodegradable), and may, for instance, comprise poly(D,L-lactide), poly(D,L-lactide-co-glycolide), poly(D,L-lactide)acid, and polyethylene glycol 3350. Put another way, in some variations, the entire drug-eluting implant (e.g., entire microsphere) may be fully bio-erodible (e.g., biodegradable). The rate of elution of a drug from an erodible implant (e.g., microsphere) described herein may be controlled by selecting an appropriate erodible material (e.g., a polymer) with predictable release characteristics (e.g., rate of release). In some embodiments, the implant (e.g., microsphere) may comprise an erodible drug-eluting matrix with variable erosion rates. For example, in some variations, a first portion of the implant may have a first erosion rate (e.g., the rate at which the drug-eluting matrix is degraded or absorbed) and a second portion of the implant may have a second, different erosion rate. Thus, elution of a drug from an implant (e.g., microsphere) described herein may have a constant or variable rate. In some embodiments, the first erosion rate may be higher than the second erosion rate, or vice versa. In some variations, the implant may comprise one or more layers, each layer comprising an erodible drug-eluting matrix. They layers may have the same erosion rates, or one or more layers may have different erosion rates. In some variations, the implant may contain a drug or erodible drug-eluting matrix. In some embodiments, the implant may comprise an erodible material (e.g., polymer), the erosion rate of which can be tuned by selecting an appropriate material (e.g., polymer). In some variations, the implant may have a first erosion rate (e.g., the rate at which outer portion of the implant is degraded or absorbed), and a drug-eluting matrix contained within may have a second erosion rate (e.g., the rate at which the drug-eluting matrix is degraded or absorbed). In some embodiments, the second erosion rate may be higher than the first erosion rate. Thus, the drug-eluting matrix may elute faster than the outer portion erodes.

### Dry Implant Formulation

As described herein, a plurality of implants may be formulated for implantation, e.g., into the eye, as a dry implant formulation, without a liquid carrier. In some variations, the dry implant formulation may be formulated as a semi-solid mass that is malleable, but does not readily dissociate into individual implants, and may be delivered together as a unit. Utilizing a dry implant formulation that is a semi-solid mass may be advantageous for a number of reasons. By way of example, a dry implant formulation formulated as a semi-solid mass may facilitate loading and/or delivery of the dry implant formulation using an implantation system (e.g., a cannula of an implantation system). In some variations, the formulation may be sufficiently malleable so as not to get clogged inside a lumen of the implantation system (e.g., the cannula). Additionally or alternatively, utilizing such a dry implant formulation may also prevent unintended or premature ejection from the implantation system (e.g., a cannula of the implantation system), due to, for example, gravity or during advancement of the cannula through tissue towards a target location.

The dry implant formulation may be endowed with a semi-solid property without use of a liquid carrier, for example through a presence of adhesion between individual implants (inter-implant adhesion). In some variations, a majority (e.g., about 51%, 60%, 65%, 70%, 75%, 80%, 85%, 90%, 95%, 96%, 97%, 98%, 99%, or 100%) of the implants of a dry implant formulation may be adhered to at least one other implant. The basis for inter-implant adhesion may be electrostatic adhesion, adhesion through a molecular intermediary, and/or through malleability of individual implants and partial deformation of the surface of one implant against one or more other implants.

In some variations, a dry implant formulation may comprise a plurality of drug-eluting implants and one or more binding agents that serve as an adhesive molecular intermediary between individual implants. In some variations, the binding agent may be selected from a sugar, a gelatin, a collagen, polyethylene glycol (PEG), a starch, a cellulose, an alginate, a chitosan, or a combination thereof.

In some variations, at least a majority of the implants may be heated above respective a glass transition temperature of the polymer or polymers comprised in the individual implants, and optionally compressed (during or shortly after heating, before the temperature of the majority of the implants fall below the respective glass transition temperatures). Glass transition temperature is the temperature at which an amorphous polymer changes from a hard/glassy state to a soft/leathery state, or vice versa Without being bound by theory, heating above the glass transition temperature, optionally combined with compression during or shortly after heating, while at least a majority of the implants are above the glass transition temperature, which may be about 1 minute, about 2 minutes, about 5 minutes, or about 10 minutes, may induce the surface of individual implants to be partially deformed against neighboring implants. In some variations, the inter-implant adhesion may be weakened in an aqueous environment, e.g., in the eye, so that individual implants may be more susceptible to dispersion after implantation.

A dose of an implant formulation (e.g., a dry implant formulation as disclosed herein) for a single implantation into the eye may be referred to herein as an “implantation dose” or “implantation unit” of the dry implant formulation. An implantation unit of an implant formulation (e.g., a dry implant formulation as disclosed herein) may comprise between about 1 and about 100, between about 100 and about 1000, between about 1000 and about 10000 or greater implants. In some embodiments, an implantation unit of an implant formulation (e.g., a dry implant formulation as disclosed herein) may comprise between about 100 and about 200, between about 100 and about 300, between about 100 and about 400, between about 100 and about 500, between about 100 and about 600, between about 100 and about 700; between about 100 and about 800, between about 100and about 900, between about 100 and about 1000, between about 200 and about 300, between about 200 and about 400, between about 200 and about 500, between about 200 and about 600, between about 200 and about 700; between about 200 and about 800, between about 200 and about 900, between about 200 and about 1000, between about 300 and about 400, between about 300 and about 500, between about 300 and about 600, between about 300 and about 700; between about 300 and about 800, between about 300 and about 900, between about 300 and about 1000, between about 400 and about 500, between about 400 and about 600, between about 400 and about 700; between about 400 and about 800, between about 400 and about 900, between about 400 and about 1000, between about 500 and about 600, between about 500 and about 700; between about 500 and about 800, between about 500 and about 900, between about 500 and about 1000, between about 600 and about 700; between about 600 and about 800, between about 600 and about 900, between about 600 and about 1000, between about 700 and about 800, between about 700 and about 900, between about 700 and about 1000, between about 800 and about 900, between about 800 and about 1000, between about 900 and about 1000, implants. In some embodiments, an implantation unit of an implant formulation (e.g., a dry implant formulation as disclosed herein) may comprise between about 1000 and about 2000, between about 1000 and about 3000, between about 1000 and about 4000, between about 1000 and about 5000, between about 1000 and about 6000, between about 1000 and about 7000; between about 1000 and about 8000, between about 1000 and about 9000, between about 1000 and about 10000, between about 2000 and about 3000, between about 2000 and about 4000, between about 2000 and 5000, between about 2000 and about 6000, between about 2000 and about 7000, between about 2000 and about 8000, between about 2000 and about 9000, between about 2000 and about 10000, between about 3000 and about 4000, between about 3000 and about 5000, between about 3000 and about 6000, between about 3000 and about 7000, between about 3000 and about 8000, between about 3000 and about 9000, between about 3000 and about 10000, between about 4000 and about 5000, between about 4000 and about 6000, between about 4000 and about 7000; between about 4000 and about 8000, between about 4000 and about 9000, between about 4000 and about 10000, between about 5000 and about 6000, between about 5000 and about 7000; between about 5000 and about 8000, between about 5000 and about 9000, between about 5000 and about 10000, between about 6000 and about 7000, between about 6000 and about 8000, between about 6000 and about 9000, between about 6000 and about 10000, between about 7000 and about 8000, between about 7000 and about 9000, between about 7000 and about 10000, between about 8000 and about 9000, between about 8000 and about 10000, between about 9000 and about 10000, implants.

A volume of an implantation unit of an implant formulation (e.g., a dry implant formulation as disclosed herein) will depend on the number and size of the implants comprised therein, as well as the amount binding agent, if any, in the formulation. With the above considerations, the volume of an implantation unit may be, for example, between about 1 μl (microliter) and about 500 μl, between about 1 μl and about 50 μl, between about 1 μl and about 100 μl, between about 1 μl and about 200 μl, between about 1 μl and about 300 μl, between about 1 μl and about 400 μl, between about 1 μl and about 40 μl, between about 2 μl and about 50 μl, between about 2 μl and about 100 μl, between about 2 μl and about 200 μl, between about 2 μl and about 300 μl, between about 2 μl and about 400 μl, between about 2 μl and about 500 μl, between about 4 μl and about 50 μl, between about 4 μl and about 100 μl, between about 4 μl and about 200 μl, between about 4 μl and about 300 μl, between about 4 μl and about 400 μl, between about 4 μl and about 500 μl, between about 10 μl and about 50 μl, between about 10 μl and about 100 μl, between about 10 μl and about 200 μl, between about 10 μl and about 300 μl, between about 10 μl and about 400 μl, between about 10 μl and about 500 μl, between about 50 μl and about 100 μl, between about 50 μl and about 200 μl, between about 50 μl and about 300 μl, between about 50 μl and about 400 μl, between about 50 μl and about 500 μl, In certain variations, an implant formulation(e.g., a dry implant formulation as disclosed herein) may be configured to elute one drug, while in other variations, an implant formulation (e.g., a dry implant formulation as disclosed herein) may be configured to elute two or more drugs. In certain variations, an implant formulation (e.g., a dry implant formulation as disclosed herein) may comprise two or more subsets of implants. In these variations, a first subset of implants may comprise a first drug and a second subset of implant may comprise a second, different drug. The first and second subsets of implants may be mixed, uniformly or non-uniformly, throughout the implant formulation, so that the combined implant formulation is capable of eluting both drugs. For example, the first subset implants may comprise a timolol (for example timolol maleate or timolol hemihydrate) and the second subset of implants may comprise a dorzolamide (for example dorzolamide hydrochloride or dorzolamide base) or brinzolamide. In another example, the first subset of implants may comprise a timolol (for example timolol maleate or timolol hemihydrate) and the second subset of implants may comprise a prostamide analog (e.g. bimatoprost) or a prostaglandin analog, e.g., latanoprost. In another example, the first subset of implants may comprise a timolol (for example timolol maleate or timolol hemihydrate) and the second subset of implants may comprise a rho-kinase inhibitor (for example ripasudil or netarsudil). In another example, the first subset of implants may comprise a prostamide analog (e.g. bimatoprost) or a prostaglandin analog, e.g., latanoprost, and the second subset of implants may comprise a rho-kinase inhibitor (for example ripasudil or netarsudil). Additionally or alternatively, in some variations, each individual implant may comprise two or more drugs (e.g., two, three, four, five, six, or more). In these variations, an implant formulation (e.g., a dry implant formulation as disclosed herein) may comprise a first subset of implants that comprise a first drug, and one or more subsets (e.g., two, three, four, five, six or more subsets) of implants that comprise one or more drugs. For example, the implant formulation (e.g., a dry implant formulation as disclosed herein) may comprise a first subset of implants that comprise a first drug and a second subset of implants in which each implant in the second subset comprises a second drug and a third drug. In some variations, the first drug may be the same as one of the second and third drugs, while in other variations, each of the first drug, the second drug, and third drug may be different drugs. In this manner, a precise amount of one or more drugs (e.g., two, three, four, five, six, or more) can be delivered together to a location of implantation. Drugs

The drug-eluting implants described herein comprise one or more drugs (e.g., two, three, four, five, or more) useful for treating the condition of the eye. In some embodiments, the condition of the eye may be glaucoma, dry eye disease, AMD, retinal diseases (e.g., retinal vascular disease), nerve disease, corneal disease, lens diseases, uvea diseases, vitreous diseases, surface diseases, lid diseases, or ocular infections. In some embodiments, the one or more drugs may comprise a drug suitable for treating glaucoma, and diseases of the retina, lens, cornea, uvea, vitreous, iris, ciliary body, sclera, or ocular surface. Such drugs include, but are not limited to:
Corticosteroids, e.g., prednisolone, prednisone, cortisone, cortisol, or triamcinolone;
anti-VEGF agents, e.g. ranibizumab, aflibercept, bevacizumab, or brolucizumab;
growth factors, e.g., a nerve growth factor, or an insulin-like growth factor;
prostaglandins or prostaglandin analogs, e.g., latanoprost, travoprost, tafluprost, or unoprostone;
prostamides or prostamide analogs, e.g. bimatoprost;
nitric oxide-releasing drugs and nitric oxide donors;
alpha-1 adrenergic agonists, e.g. or epinephrine, dipivefrin;
alpha-2 adrenergic agonists, e.g. epinephrine, dipivefrin, brimonidine, or apraclonidine;
beta-adrenergic blockers “beta blockers”, e.g., a levobunolol, a timolol, a betaxolol, a carteolol, or a metipranolol;
miotics, e.g., pilocarpine;
carbonic anhydrase inhibitors, e.g. an acetazolamide, a methazolamide, a dorzolamide, a brinzolamide, a diclofenamide, a ethoxzolamide, or a zonisamide;
Rho-kinase (ROCK) inhibitors, e.g., ripasudil or netarsudil;
Parasympathomimetic;
Sympathomimetics, e.g., muscarinic antagonists (e.g., atropine);
antimetabolites, e.g., fluoruracil mitomycin C;
antibiotics;
non-steroidal anti-inflammatory drugs, e.g., an NSAID;
antifungals;
immunosuppressants e.g., cyclosporine, sirolimus, everolimus, or tacrolimus;
vitreous hemorrhage drugs;
collagenases;
vitreous floater treatments;
pupil dilators;
pupil constrictors;
anticoagulants (e.g., heparin);
fibrinolytic compounds;
monoclonal antibodies or other biologics;
and combinations thereof.

In certain embodiments, the one or more drugs is a nitric oxide-releasing drug in combination with a prostaglandin/prostaglandin analog or other glaucoma drug (e.g., to target multiple mechanisms of action). In certain variations, the drug is useful for lowering intraocular pressure. In certain variations, the drug may suppress production of aqueous humor. In some variations, the drug may increase the drainage of aqueous humor through a trabeculocanalicular pathway and/or a uveoscleral pathway.

The amount of drug within a given implant (e.g., microsphere or microparticle) or a given dose of a plurality of implants, e.g., an implantation unit of an implant formulation(e.g., a dry implant formulation as disclosed herein), can be adjusted depending on the type of drug and/or application. For instance, an implant (e.g., microparticle implant or microsphere) or an implantation unit of an implant formulation (e.g., a dry implant formulation as disclosed herein) may comprise between about 1 μg and about 500 μg of a drug, or between about 30 ng and about 90 mg of a drug. In some variations, an implant or an implantation unit of an implant formulation (e.g., a dry implant formulation as disclosed herein) may have between about 1 μg and about 400 μg, between about 1 μg and about 300 μg, between about 1 μg and about 200 μg, between about 1 μg and about 100 μg, between about 1 μg and about 10 μg, between about 1 μg and about 5 μg, between about 5 μg and about 10 μg, between about 5 μg and about 100 μg, between about 10 μg and about 100 μg, between about 10 μg and about 90 μg, between about 10 μg and about 80 μg, between about 10 μg and about 70 μg, between about 10 μg and about 60 μg, between about 10 μg and about 50 μg, between about 10 μg and about 40 μg, between about 10 μg and about 30 μg, between about 10 μg and about 20 μg, between about 20 μg and about 100 μg, between about 20 μg and about 90 μg, between about 20 μg and about 80 μg, between about 20 μg and about 70 μg, between about 20 μg and about 60 μg, between about 20 μg and about 50 μg, between about 20 μg and about 40 μg, between about 20 μg and about 30 μg, between about 40 μg and about 100 μg, between about 30 μg and about 90 μg, between about 30 μg and about 80 μg, between about 30 μg and about 70 μg, between about 30 μg and about 60 μg, between about 30 μg and about 50 μg, between about 30 μg and about 40 μg, between about 40 μg and about 100 μg, between about 40 μg between and about 90 μg, about 40 μg and about 80 μg, between about 40 μg and about 70 μg, between about 40 μg and about 60 μg, between about 40 μg and about 50 μg, between about 50 μg and about 100 μg, between about 50 μg and about 90 μg, between about 50 μg and about 80 μg, between about 50 μg and about 70 μg, between about 50 μg and about 60 μg, between about 60 μg and about 100 μg, between about 60 μg and about 90 μg, between about 60 μg and about 80 μg, between about 60 μg and about 70 μg, between about 70 μg and about 100 μg, between about 70 μg and about 90 μg, between about 70 μg and about 80 μg, between about 80 μg and about 100 μg, between about 80 μg and about 90 μg; or between about 90 μg and about 100 μg of drug (including all sub-ranges and values therein). In some embodiments, an implant (e.g., microsphere), or an implantation unit of an implant formulation (e.g., a dry implant formulation as disclosed herein) may have about 1, 2, 3, 4, 5, 6, 7, 8, 9, 10, 15, 20, 30, 40, 50, 60, 70, 80, 90, 100, 150, 200, 250, 300, 350, 400, 450, or 500 μg of a drug. In some variations, an implant or an implantation unit of an implant formulation (e.g., a dry implant formulation as disclosed herein) may have between about 30 ng and about 90 mg, between about 30 ng and about 80 mg, between about 30 ng and about 70 mg, between about 30 ng and about 60 mg, between about 30 ng and about 50 mg, between about 30 ng and about 40 mg, between about 30 ng and about 30 mg, between about 30 ng and about 20 mg, between about 30 ng and about 10 mg, between about 30 ng and about 5 mg, between about 30 ng and about 1 mg, between about 30 ng and about 500 μg, between about 30 ng and about 100 μg, between about 30 ng and about 900 ng, between about 30 ng and about 800 ng, between about 30 ng and about 700 ng, between about 30 ng and about 600 ng, between about 30 ng and about 500 ng, between about 30 ng and about 400 ng, between about 30 ng and about 300 ng, between about 30 ng and about 200 ng, between about 30 ng and about 100 ng, between about 30 ng and about 50 ng, between about 100 ng and about 90 mg, between about 100 ng and about 80 mg, between about 100 ng and about 70 mg, between about 100 ng and about 60 mg, between about 100 ng and about 50 mg, between about 100 ng and about 40 mg, between about 100 ng and about 30 mg, between about 100 ng and about 20 mg, between about 100 ng and about 10 mg, between about 100 ng and about 5 mg, between about 100 ng and about 1 mg, between about 100 ng and about 500 μg, between about 100 ng and about 100 μg, between about 200 ng and about 90 mg, between about 200 ng and about 80 mg, between about 200 ng and about 70 mg, between about 200 ng and about 60 mg, between about 200 ng and about 50 mg, between about 200 ng and about 40 mg, between about 200 ng and about 30 mg, between about 200 ng and about 20 mg, between about 200 ng and about 10 mg, between about 200 ng and about 5 mg, between about 200 ng and about 1 mg, between about 200 ng and about 500 μg, between about 200 ng and about 100 μg, between about 200 ng and about 900 ng, between about 200 ng and about 800 ng, between about 200 ng and about 700 ng, between about 200 ng and about 600 ng, between about 200 ng and about 500 ng, between about 200 ng and about 400 ng of drug (including all sub-ranges and values therein).

In some variations, an implant or a plurality of implants, e.g., an implantation unit of an implant formulation (e.g., a dry implant formulation as disclosed herein), may have between about 1 ug and about 90 mg, between about 1 μg and about 70 mg, between about 1 μg and about 50 mg, between about 1 μg and about 40 mg, between about 1 μg and about 30 mg, between about 1 μg and about 20 mg, between about 1 μg and about 15 mg, between about 1 μg and about 10 mg, between about 1 μg and about 5 mg, between about 1 μg and about 1 mg, between about 5 μg and about 90 mg, between about 5 μg and about 70 mg, between about 5 μg and about 50 mg, between about 5 μg and about 40 mg, between about 5 μg and about 30 mg, between about 5 μg and about 20 mg, between about 5 μg and about 15 mg, between about 5 μg and about 10 mg, between about 5 μg and about 5 mg, between about 5 μg and about 1 mg, between about 10 μg and about 90 mg, between about 10 μg and about 70 mg, between about 10 μg and about 50 mg, between about 10 μg and about 40 mg, between about 10 μg and about 30 mg, between about 10 μg and about 20 mg, between about 10 μg and about 15 mg, between about 10 μg and about 10 mg, between about 10 μg and about 5 mg, between about 10 μg and about 1 mg, between about 20 μg and about 90 mg, between about 20 μg and about 70 mg, between about 20 μg and about 50 mg, between about 20 μg and about 40 mg, between about 20 μg and about 30 mg, between about 20 μg and about 20 mg, between about 20 μg and about 15 mg, between about 20 μg and about 10 mg, between about 20 μg and about 5 mg, between about 20 μg and about 1 mg, between about 40 μg and about 90 mg, between about 40 μg and about 70 mg, between about 40 μg and about 50 mg, between about 40 μg and about 40 mg, between about 40 μg and about 30 mg, between about 40 μg and about 20 mg, between about 40 μg and about 15 mg, between about 40 μg and about 10 mg, between about 40 μg and about 5 mg, between about 40 μg and about 1 mg, between about 60 μg and about 90 mg, between about 60 μg and about 70 mg, between about 60 μg and about 50 mg, between about 60 μg and about 40 mg, between about 60 μg and about 30 mg, between about 60 μg and about 20 mg, between about 60 μg and about 15 mg, between about 60 μg and about 10 mg, between about 60 μg and about 5 mg, between about 60 μg and about 1 mg, between about 80 μg and about 90 mg, between about 80 μg and about 70 mg, between about 80 μg and about 50 mg, between about 80 μg and about 40 mg, between about 80 μg and about 30 mg, between about 80 μg and about 20 mg, between about 80 μg and about 15 mg, between about 80 μg and about 10 mg, between about 80 μg and about 5 mg, between about 80 μg and about 1 mg, between about 100 μg and about 90 mg, between about 100 μg and about 70 mg, between about 100 μg and about 50 mg, between about 100 μg and about 40 mg, between about 100 μg and about 30 mg, between about 100 μg and about 20 mg, between about 100 μg and about 15 mg, between about 100 μg and about 10 mg, between about 100 μg and about 5 mg, or between about 100 μg and about 1 mg of drug (including all sub-ranges and values therein).

In some embodiments, an implant (e.g., microsphere) or a plurality of implants, (e.g. an implantation unit of an implant formulation (e.g., a dry implant formulation as disclosed herein) as described herein) may have about 30, 40, 50, 60, 70, 80, 90, 100, 150, 200, 250, 300, 350, 400, 450, 500, 600, 700, 800, 900, or 1000 ng of a drug, or about 1, 2, 3, 4, 5, 6, 7, 8, 9, 10, 15, 20, 30, 40, 50, 60, 70, 80, 90, 100, 150, 200, 250, 300, 350, 400, 450, 500, 600, 700, 800, 900, or 1000 μg of a drug, or about 1, 2, 3, 4, 5, 6, 7, 8, 9, 10, 15, 20, 30, 40, 50, 60, 70, 80, or 90 mg of a drug,

The implants of this disclosure may elute drugs at a rate determined by their composition, size, and/or implant location. Thus, a proper dosage of drug may be administered to the eye by adjusting the properties of the implants (e.g., microparticle implants or microspheres). In some embodiments of the implants described herein, the drug-eluting implant or an implantation unit of an implant formulation (e.g., a dry implant formulation as disclosed herein) may deliver one or more drugs into the eye at a rate of between about 1 ng/day and about 3000 ng/day, between about 1 mg/day and about 300 mg/day, or between about 1 ng/day and about 300 mg/day. In certain embodiments, the drug-eluting implant or implantation unit of an implant formulation (e.g., a dry implant formulation as disclosed herein) may deliver a drug into the eye at a rate of between about 1 ng/day and about 2000 ng/day, between about 1 ng/day and about 1000 ng/day, between about 1 ng/day and about 500 ng/day, between about 1 ng/day and about 400 ng/day, between about 1 ng/day and about 300 ng/day, between about 1 ng/day and about 200 ng/day, between about 1 ng/day and about 100 ng/day, between about 1 ng/day and about 50 ng/day, between about 5 ng/day and about 3000 ng/day or more, between about 5 ng/day and about 2000 ng/day, between about 5 ng/day and about 1000 ng/day, between about 5 ng/day and about 500 ng/day, between about 5 ng/day and about 400 ng/day, between about 5 ng/day and about 300 ng/day, between about 5 ng/day and about 200 ng/day, between about 5 ng/day and about 100 ng/day, between about 5 ng/day and about 50 ng/day, between about 10 ng/day and about 3000 ng/day or more, between about 10 ng/day and about 2000 ng/day, between about 10 ng/day and about 1000 ng/day, between about 10 ng/day and about 500 ng/day, between about 10 ng/day and about 400 ng/day, between about 10 ng/day and about 300 ng/day, between about 10 ng/day and about 200 ng/day, between about 10 ng/day and about 100 ng/day, between about 10 ng/day and about 50 ng/day, between about 50 ng/day and about 3000 ng/day or more, about 50 ng/day and about 2000 ng/day, between about 50 ng/day and about 1000 ng/day, between about 50 ng/day and about 500 ng/day, between about 50 ng/day and about 400 ng/day, between about 50 ng/day and about 300 ng/day, between about 50 ng/day and about 200 ng/day, between about 50 ng/day and about 100 ng/day, between about 100 ng/day and about 3000 ng/day or more, between about 100 ng/day and about 2000 ng/day, between about 100 ng/day and about 1000 ng/day, between about 100 ng/day and about 500 ng/day, between about 100 ng/day and about 400 ng/day, between about 100 ng/day and about 300 ng/day, between about 100 ng/day and about 200 ng/day, about 200 ng/day to 300 ng/day, about 300 ng/day to 400 ng/day, about 400 ng/day to 500 ng/day, about 500 ng/day to 1000 ng/day, about 1000 ng/day to 1500 ng/day, about 1500 ng/day to 2000 ng/day, about 2000 ng/day to 2500 ng/day, or about 2500 ng/day to 3000 ng/day, (including all sub-ranges and values of any of the above). In certain embodiments, the drug-eluting implant or implantation unit of an implant formulation (e.g., a dry implant formulation as disclosed herein) may deliver one or more drugs into the eye at a rate of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, or 3000 ng/day.

In certain embodiments, the drug-eluting implant or implantation unit of an implant formulation (e.g., a dry implant formulation as disclosed herein) may deliver a drug into the eye at a rate of between about 1 mg/day and about 300 mg/day, between about 1 mg/day and about 200 mg/day, between about 1 mg/day and about 100 mg/day, between about 1 mg/day and about 50 mg/day, between about 1 mg/day and about 20 mg/day, between about 1 mg/day and about 10 mg/day, between about 10 mg/day and about 300 mg/day, between about 10 mg/day and about 200 mg/day, between about 10 mg/day and about 100 mg/day, between about 10 mg/day and about 50 mg/day, between about 10 mg/day and about 20 mg/day, between about 100 mg/day and about 300 mg/day, between about 100 mg/day and about 200 mg/day (including all sub-ranges and values of any of the above). In certain embodiments, the drug-eluting implant or implantation unit of an implant formulation (e.g., a dry implant formulation as disclosed herein) may deliver one or more drugs into the eye at a rate of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, or 300 mg/day.

In certain embodiments, the drug-eluting implant or implantation unit of an implant formulation(e.g., a dry implant formulation as disclosed herein) may deliver a drug into the eye at a rate of between about 1 ng/day and about 300 mg/day, between about 1 ng/day and about 200 mg/day, between about 1 ng/day and about 100 mg/day, between about 1 ng/day and about 50 mg/day, between about 1 ng/day and about 20 mg/day, between about 1 ng/day and about 10 mg/day, between about 10 ng/day and about 300 mg/day, between about 10 ng/day and about 200 mg/day, between about 10 ng/day and about 100 mg/day, between about 10 ng/day and about 50 mg/day, between about 10 ng/day and about 20 mg/day, between about 10 ng/day and about 10 mg/day, between about 10 ng/day and about 1 mg/day, between about 20 ng/day and about 200 mg/day, between about 20 ng/day and about 100 mg/day, between about 20 ng/day and about 50 mg/day, between about 20 ng/day and about 20 mg/day, between about 20 ng/day and about 10 mg/day, between about 20 ng/day and about 1 mg/day, between about 30 ng/day and about 200 mg/day, between about 30 ng/day and about 100 mg/day, between about 30 ng/day and about 50 mg/day, between about 30 ng/day and about 20 mg/day, between about 30 ng/day and about 10 mg/day, between about 30 ng/day and about 1 mg/day, between about 50 ng/day and about 200 mg/day, between about 50 ng/day and about 100 mg/day, between about 50 ng/day and about 50 mg/day, between about 50 ng/day and about 20 mg/day, between about 50 ng/day and about 10 mg/day, between about 50 ng/day and about 1 mg/day, between about 100 ng/day and about 300 mg/day, between about 100 ng/day and about 200 mg/day, between about 100 ng/day and about 100 mg/day, between about 100 ng/day and about 50 mg/day, between about 100 ng/day and about 20 mg/day, between about 100 ng/day and about 10 mg/day, between about 100 ng/day and about 1 mg/day, between about 200 ng/day and about 300 mg/day, between about 200 ng/day and about 200 mg/day, between about 200 ng/day and about 100 mg/day, between about 200 ng/day and about 50 mg/day, between about 200 ng/day and about 20 mg/day, between about 200 ng/day and about 10 mg/day, or between about 200 ng/day and about 1 mg/day (including all sub-ranges and values of any of the above).

As described below, drugs suitable for delivery by the implants described herein may diffuse from the location of implantation in one part/location of the eye (e.g., vitreous, subconjunctival space) to another part/location in the eye (e.g., anterior chamber, posterior chamber), or vice versa. To enhance this diffusion from the implant location to another location in the eye, the drugs described herein may be administered together with the application of one or more penetration enhancers. Penetration enhancers may include, for instance, compounds such as cyclodextrins, chelating agents, crown ethers, bile acids, bile salts, surfactants, cell-penetrating peptides, and amphiphilic compounds. Such penetration enhancers may be combined with the drug to treat a condition of the eye being administered by a drug-eluting implant described herein, or they may be administered separately. In some variations, a first drug-eluting implant delivers a drug to treat a condition of the eye, and a second drug-eluting implant delivers a penetration enhancer. In other variations, one drug-eluting implant delivers both a drug to a treat a condition of the eye and a penetration enhancer. Penetration enhancers may also be non-compound penetration enhancers, which are applied separately and in addition to the implant. For instance, a non-compound penetration enhancer may include electrical currents, iontophoresis, ultrasound, or microneedles. These may, for instance, be applied to a tissue of the eye to increase penetration of a drug delivery by a drug-eluting implant described herein. Selection of the appropriate penetration enhancer may depend upon the properties of the drug being administered (e.g., molecular weight, hydrophobicity/lipophilicity). An appropriate penetration enhancer may be selected for enhancing penetration of a drug through a specific tissue (e.g., cornea, sclera).

Currently available treatments for eye disorders such as glaucoma use medicated drops that persist for less than 24 hours. For age-related macular degeneration (AMD) and other retinal diseases, certain other ocular injections provide delivery of drugs to eye tissues for a few weeks or a few months. In contrast, the drug-eluting implants described herein allow for the continuous or semi-continuous delivery of one or more drugs for days or weeks, and/or months or years. In some embodiments, the drug is delivered to the eye over a predetermined period of time, such as, for example, over at least about 1, 2, 3, 4, 5, 6, 7, 8, 9, 10, 12, 18, 24, 30, 36, 48, 60, or 72 months (including all values and sub-ranges therein). For example, in some variations, the drug-eluting implant is configured to for sustained-release of drug to the eye for between about 1 month and about 3 months, about 2 months and about 4 months, about 1 month and about 6 months, about 6 months and about 9 months, about 6 months and about 12 months, about 12 months and about 18 months, about 12 months and about 24 months, about 24 months and about 36 months, about 12 months to about 72 months or about 1 month to about 72 months. In certain embodiments, the period of time is from between about 0 months and 1 month, between about 0 months and about 2 months, between about 0 months and about 3 months, between about 0 months and about 4 months, between about 0 months and about 5 months, between about 0 months and about 6 months, between about 0 months and about 7 months, between about 0 months and about 8 months, between about 0 months and about 9 months, between about 0 months and about 10 months, between about 0 months and about 11 months, between about 0 months and about 12 months, between about 1 month and about 2 months, between about 1 month and about 3 months, between about 1 month and about 4 months, between about 1 month and about 5 months, between about 1 month and about 6 months, between about 1 month and about 7 months, between about 1 month and about 8 months, between about 1 month and about 9 months, between about 1 month and about 10 months, between about 1 month and about 11 months, between about 1 month and about 12 months, between about 2 months and about 3 months, between about 2 months and about 4 months, between about 2 months and about 5 months, between about 2 months and about 6 months, between about 2 months and about 7 months, between about 2 months and about 8 months, between about 2 months and about 9 months, between about 2 months and about 10 months, between about 2 months and about 11 months, between about 2 months and about 12 months, between about 3 months and about 4 months, between about 3 months and about 5 months, between about 3 months and about 6 months, between about 3 months and about 7 months, between about 3 months and about 8 months, between about 3 months and about 9 months, between about 3 months and about 10 months, between about 3 months and about 11 months, between about 3 months and about 12 months, between about 4 months and about 5 months, between about 4 months and about 6 months, between about 4 months and about 7 months, between about 4 months and about 8 months, between about 4 months and about 9 months, between about 4 months and about 10 months, between about 4 months and about 11 months, between about 4 months and about 12 months, between about 5 months and about 6 months, between about 5 months and about 7 months, between about 5 months and about 8 months, between about 5 months and about 9 months, between about 5 months and about 10 months, between about 5 months and about 11 months, between about 5 months and about 12 months, between about 6 months and about 7 months, between about 6 months and about 8 months, between about 6 months and about 9 months, between about 6 months and about 10 months, between about 6 months and about 11 months, between about 6 months and about 12 months, between about 7 months and about 8 months, between about 7 months and about 9 months, between about 7 months and about 10 months, between about 7 months and about 11 months, between about 7 months and about 12 months, between about 8 months and about 9 months, between about 8 months and about 10 months, between about 8 months and about 11 months, between about 8 months and about 12 months, between about 9 months and about 10 months, between about 9 months and about 11 months, between about 9 months and about 12 months, between about 10 months and about 11 months, between about 10 months and about 12 months, between about 11 months and about 12 months, between about 10 months and about 24 months, between about 10 months and about 36 months, between about 12 months and about 24 months, or between about 12 months and about 36 months. In certain embodiments, the period of time is at least about 1 year, 2 years, 3 years, 4 years, 5 years, 10 years, 15 years, 20 years or between about 1 year and about 5 years, about 1 year and about 10 years, about 5 years and about 10 years, about 5 years and about 15 years, about 10 years and about 20 years, about 5 years and about 20 years, or about 1 year and about 20 years. As used above, “ about 0 months” refers to the time of approximate implantation of one implant or a plurality of implants.

In some variations, multiple implants or a plurality of subsets of implants (for example, in an implant formulation) may have a similar or the same delivery periods. In some variations, multiple implants or a plurality of subsets of implants (for example, in an implant formulation) may have independent delivery periods (e.g., the predetermined periods of time as described above). By way of example only, two subsets of implants (for example, in an implant formulation) may be administered to a subject, wherein the first subset has a period of time of between about 0 months and about 3 months, and the second subset has a period of time of between about 4 months and about 9 months. It is to be understood that each implant or subset of implants may independently have any of the delivery periods described above.

As discussed, the drug-eluting implants described herein may be at least partly bio-erodible. In some variations, one or more new implants (e.g., replacement implants) may be delivered to one or more locations of the eye when one or more implanted implants degrade within the eye. For instance, a new implant may replace a partially or fully degraded implant every month, every 2 months, every 3 months, every 6 months, every 12 months, every 18 months, every 2 years, every 3 years, or more, or at any interval therein.

As discussed previously, in variations in which the implant (e.g., microparticle such as a microsphere) is positioned at least partially within the posterior chamber (e.g., sulcus, remainder of the posterior chamber), the drug delivered from the drug-eluting implant may be taken up by anterior and/or posterior flowing currents such that it may be delivered to the anterior and/or posterior chambers of the eye. In some variations, the drug may be delivered to the anterior chamber, the posterior chamber (e.g., the sulcus and/or remainder of the posterior chamber), the cornea, the iris, the lens, the pupil, the retina, or the vitreous body. In variations in which the implant is positioned intramurally (e.g., embedded or positioned entirely within a tissue or tissues of the eye), the drug may be delivered through the sclera to the anterior chamber, through the sclera to the posterior chamber, through the cornea to the anterior chamber, through the cornea to the posterior chamber, through the limbus to the anterior chamber, or through the limbus to the posterior chamber. In some variations, the drug may be delivered through the conjunctiva to the subconjunctival space or through the conjunctiva and Tenon's capsule to the sub-Tenon's space. In some variations, the implant is positioned in the sulcus, and posterior currents deliver the eluted drug to the retina and/or choroid.

It may be advantageous for the implants (e.g., microparticles such as microspheres) described herein to comprise at least one imaging agent that may assist in the visualization of the implant(s) and/or drug during and/or after implantation. In variations comprising an imaging agent, the imaging agent may be released as the bio-erodible implant degrades, which may further assist in visualizing and/or quantifying how much drug of the initial implant dose remains at any given point in time and/or over a period of time (e.g., days, weeks, years). In other words, the imaging agent acts as a proxy for drug elution. In some variations, the imaging agent may be one or more of a dye, a radiolabel, and a fluorescent marker. In certain embodiments, the imaging agent may be fluorescein. In some variations of the implants described herein, an implant may comprise a drug and an imaging agent and the implant may be configured to deliver the drug and the imaging agent to the eye at the same delivery rate (e.g., elution rate). In other variations, the implant may be configured to deliver the drug and the imaging agent to the eye at different delivery rates. In some instances, a medical professional may visualize the imaging agent to estimate or otherwise quantify the amount of drug delivered, and may personalize treatment based on this determination. For example, the medical profession may determine a characteristic of a future procedure (e.g., number or volume of implants, type of drug, amount of drug, time for a next implantation, amount of time a next implant(s) should remain in the eye) based on the visualization data of the imaging agent and/or the estimate or quantification of the amount of drug delivered.

The implants (e.g., microparticles such as microspheres) described herein may be configured for placement within the eye. For example, in some variations, the microsphere may reside partially or wholly in the subconjunctival space, the Tenon's capsule, the sub-Tenon's space, the anterior chamber (including the iridocorneal angle), the posterior chamber (including the sulcus), or the vitreous.

### Implant Locations

The drug-eluting implants described herein may be configured to reside in any suitable location in the eye. For example, the implants described herein may reside in the subconjunctival space, the Tenon's capsule, the sub-Tenon's space, the suprachoroidal space (which can be accessed for implantation via an ab externo approach or ab interno approach through the anterior chamber angle), the subretinal space, the sclera, the cornea, the limbus, the anterior chamber, the posterior chamber (including the sulcus and the remainder of the posterior chamber), and the vitreous.

Implants may be delivered to multiple, different implantation sites. In these instances, the implants in one portion of the eye may have one or more different drugs and/or may have different delivery rates (e.g., elution rates, dissolution rates) than the implants in another portion of the eye. By way of example, a first implant or first subset of implants may be configured to reside in a first location, and a second implant or second subset of implants may be configured to reside in a second location. In some embodiments, a first implant or first subset of implants may be configured to reside in a first location may comprise a first drug, and a second implant or second subset of implants configured to reside in a second location may comprise the same or a different drug. In some embodiments, a first implant or first subset of implants may be configured to reside in a first location may have a first drug delivery rate, and a second implant or second subset of implants configured to reside in a second location may have the same or a different drug delivery rate.

Drug-eluting implants may also reside fully intramurally (e.g., embedded or positioned entirely within a tissue or tissues of the eye), such as, for example, the cornea, sclera, limbus, or a combination thereof. In certain variations, one or more implants may reside fully within the cornea, fully within the sclera, or fully within the limbus.

### Implantation System

As described herein, an implant and/or an implant formulation (e.g., a dry implant formulation as disclosed herein) may be implanted, e.g., into the eye, using an implantation system configured to access a target location of an eye of the subject, and implant one or more implants, or a desired amount, e.g. an implantation unit, of the implant formulation described herein into the target location. In some embodiments, the implantation system may be configured to implant the implant or a dry implant formulation as disclosed herein without a carrier.

In certain variations, the implantation system may comprise an implantation device comprising a handle and a cannula coupled to a distal end of the handle. The handle may be sized and shaped for a user to comfortably hold and manipulate the cannula to advance the cannula or a portion thereof towards a target location in the eye. In some variations, the lumen of the cannula or a portion thereof may be loaded (e.g., pre-loaded) with one or more implants, such as, for example, an implantation unit of an implant formulation (e.g., a dry implant formulation as disclosed herein). In some variations, the implantation system may further comprise a plunger or a pusher rod that is slidably positioned within the lumen of the cannula. In some variations, the handle may comprise an actuator for the user to control the implantation system (e.g., control the plunger or pusher rod) to eject the implant formulation from the cannula, in order to deliver the implant(s) to the target location in the eye.

The cannula may comprise a distal portion configured for insertion into a target location in the eye. The cannula may be made of stainless steel, metal, Teflon, or a polymer (e.g., a polycarbonate, a polyethylene, a polyamide, or a polyether ether ketone). The distal portion of the cannula may comprise and terminate at a distal tip, where the lumen terminates as a distal opening in the distal tip. The one or more implants, such as, for example, an implant formulation (e.g., a dry implant formulation as disclosed herein), may be positioned within the lumen of the cannula. The distal tip may be sufficiently sharp to penetrate tissue and/or membranes in the eye. By way of example, the distal tip of the cannula may have a beveled edge. Additionally or alternatively, the distal portion comprising the distal tip may be tapered, so that the outer diameter of the cannula gradually decreases towards the distal tip, and the external diameter of the distal tip is less than the external diameter of the proximal portion of the cannula. In some variations, the cannula is flexible. In some variations, the cannula is transparent. A transparent cannula advantageously provides for visualization of the implants or the implant formulation (e.g., a dry implant formulation as disclosed herein) loaded in the lumen, and visual control of the implantation process.

In some variations, the implantation device may be a syringe that is operatively coupled to a cannula loaded with one or more implants or an implant formulation (e.g., a dry implant formulation as disclosed herein), and the actuator may be a plunger of the syringe.

In some variations, the actuator may be or may comprise one or more of a wheel, a switch, a button, knob, lever, a slider, a touchpad, a capacitive touch sensor, or the like. In some variations, a distal tip of the pusher rod or plunger may be in contact with one or more implants or at least a portion of the implant formulation (e.g., a dry implant formulation as disclosed herein). In some variations, the actuator may have markings or colorings to indicate degree of advancement or direction of advancement.

In some variations, the actuator may be operatively coupled to the plunger or pusher rod, and may be operable to move the pusher rod with respect to the handle and the cannula, thereby advancing the tip of the pusher rod towards a distal opening of the cannula. In some variations, the actuator may be operatively coupled to the cannula, and may be operable to move the cannula with respect to the handle and the pusher rod, thereby retracting the distal opening of the cannula towards the distal tip of the pusher rod. In some variations, the actuator may be operatively coupled to the plunger or pusher rod and to the cannula. The actuator may be operable to move the pusher rod and the cannula in opposite directions. That is, the actuator may be operable to move the pusher rod with respect to the handle and the cannula, thereby advancing the tip of the pusher rod towards a distal opening of the cannula, as well as operable to move the cannula with respect to the handle and the pusher rod, thereby retracting the distal opening of the cannula towards the tip of the pusher rod.

In some variations, the handle may comprise a drive assembly that translates rotational movement into a linear motion of the pusher rod, the cannula, or both. For example, in some variations, the actuator may be a wheel rotatably coupled to one or more components of the drive assembly, and rotation of the wheel by the user may rotate one or more components (e.g., circular gears) of the drive assembly, which may result in linear movement of the pusher rod as will be described in more detail herein.

For example, the drive assembly may include at least one elongate member (e.g., a linear gear, which may be referred to as a “rack”) and at least one pinion gear. The at least one elongate member may be connected to the pusher rod and/or the cannula. The elongate member may be configured to engage with the pinion gear, so that rotational motion of the pinion gear is translated to linear motion of the elongate member. In some variations, the elongate member may be a linear gear, which may have teeth on its surface that engage corresponding teeth on the pinion gear. The drive assembly may have one or more idler gears that engage with the elongate member and/or the pinion gear to provide the desired direction of movement for the elongate member with respect to the direction of rotation of the pinion gear. The at least one pinion gear may also be coupled (e.g., coaxially or tangentially) to the wheel. Such coupling may be accomplished with, for example, a pin that can be coupled to (e.g., via threads or the like) a central opening in the rotatable component and pinion gear. A mechanical or other fastener (e.g., a nut) may be used to secure the rotatable component and pinion gear in a manner so that rotation of the rotatable component also rotates the pinion gear and vice versa. The wheels may be attached to the pinion gear in any suitable manner. For example, in some variations, the wheels may be positioned (e.g., slid) onto the pinion gear and may be secured thereto (e.g., with adhesive or other mechanical fastening technique such as, for example, a compression fit, press fit, or the like). In other variations, the wheels and pinion gears may be integrally formed (e.g., molded as one part using plastic injection molding technology). 2) Regardless of how the wheels may be coupled to the pinion gear, the wheels and pinion gear may rotate coaxially or tangentially, in the same direction, and at the same angular rate.

In some variations, the cannula and the pusher rod may be configured to move linearly in opposite directions, upon engagement of the actuator. In these variations, the drive assemble may comprise a first pinion gear coupled (e.g., coaxially or tangentially) to the actuator (e.g. a wheel) and configured to engage with a first elongate member connected to the pusher rod, and a second pinion gear coupled (e.g., coaxially or tangentially) to the actuator and configured to engage with a second elongate member connected to the cannula. Moreover, the drive assembly may comprise an idler gear either between the first pinion gear and the first elongate member, or between the second pinion gear and the second elongate member, so that the first and second elongate members may be moved in opposite directions when the actuator is moved (e.g., the wheel is turned) in a given direction.

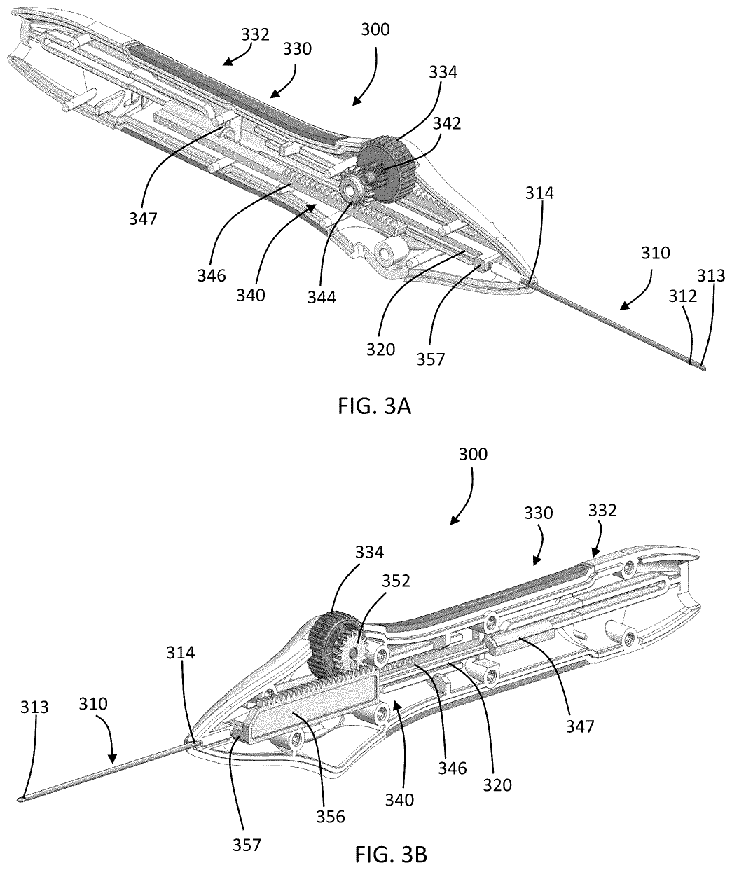

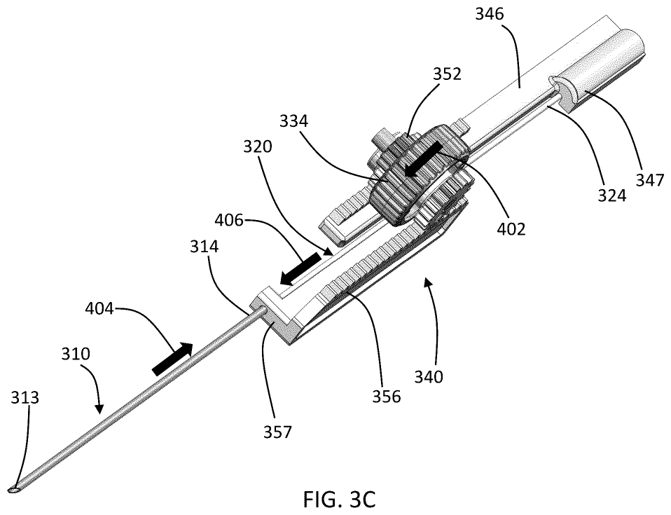

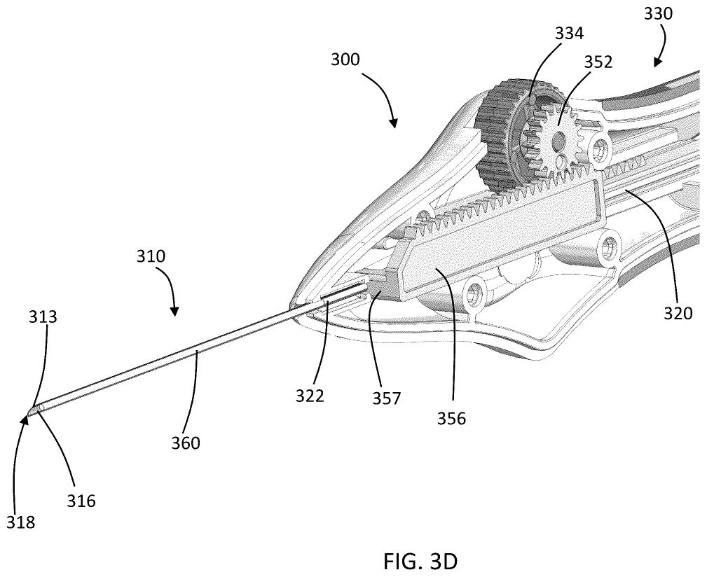

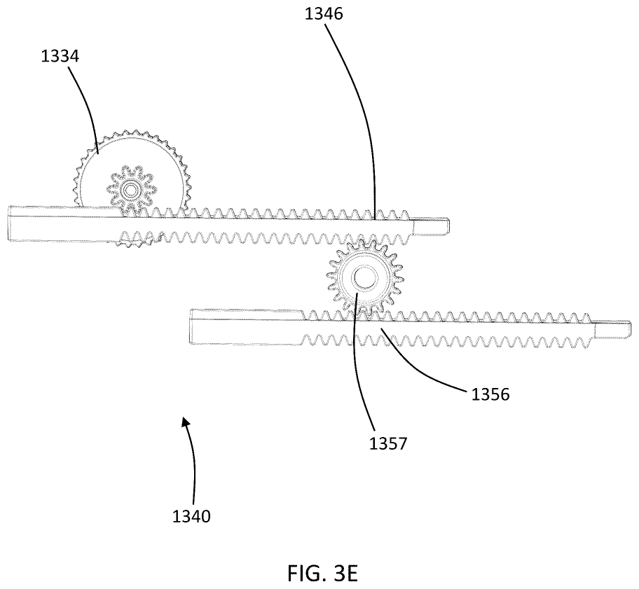

Turning now to FIGS. 3A-3B, shown there is an implantation system 300 configured to deliver an implant or an implant formulation (e.g., a dry implant formulation as disclosed herein). In the variation shown in FIGS. 3A-3B, the implantation system 300 is configured for movement of both the cannula and the pusher rod during an implantation procedure. In this variation, the implantation system 300 may comprise a cannula 310, a pusher rod 320, and a handle 330. The cannula 310 may comprise a distal portion 312, a distal tip 313 having a beveled edge, and a proximal portion 314. The pusher rod 320 may be slidably positioned in the lumen of the cannula 310. The handle 330 may comprise a housing 332 shaped to be comfortably held by a user (not shown) (e.g., in a single handle of the user), and an actuator 334 in the form of a wheel that serves to move the cannula 310 and the pusher rod 320 in opposite directions when actuated (e.g., turned) by the user. In addition, handle 330 may further comprises (e.g., at least partially house therein) a drive assembly 340 that translates the movement (e.g., rotational movement) of the actuator 334 with opposing linear movements of the cannula 310 and the pusher rod 320. The housing 332 of the handle 330 is depicted partially cut away to provide a view of the drive assembly 340 and its components, which in this variation, are housed entirely within an interior portion of the housing 332 of the handle 330.

FIG. 3C shows a top view of the implantation system 300 of FIGS. 3A and 3B with the housing 332 of the handle 330 removed to provide a better view of the drive assembly 340 and it' connection to the cannula 310 and the pusher rod 320. As can be more readily seen in this figure, a proximal portion 324 of the pusher rod 320 may be fixedly connected to the linear gear 346 via pusher rod holder 347, the proximal portion 314 of the cannula 310 may be fixedly connected to the linear gear 356 via the handle 357, and a distal portion of the pusher rod 320 may be slidably positioned in a lumen of cannula 310.

FIG. 3E shows another variation of a drive assembly 1340 that may be housed in the housing 332, that translates the movement (e.g., rotational movement) of an actuator (e.g. actuator 1334) with opposing linear movements of the cannula (not shown) and the pusher rod (not shown), for delivery of an implant or implant formulation (e.g. a dry implant formulation) loaded in the cannula. In this variation, the drive assembly 1340 may comprise a first linear gear 1346 fixedly coupled to a pusher rod (not shown), and a second linear gear 1356 fixedly coupled to the cannula (not shown). The first linear gear 1346 and the second linear gear 1356 may be engaged with one another via an idler gear 1357. As such, the drive assembly 1340 may be configured to move the pusher rod (via the first linear gear 1346) and the cannula (via the second linear gear 1356) in opposite linear directions.

The implantation system 330 may be configured so that when, the cannula 310 is retracted and pusher rod 320 is advanced through engagement (e.g. rotation) of actuator 334, the dry implant formulation 360 is ejected through a distal opening 318 of the lumen 316. FIG. 3D is a close-up view of a distal portion of the implantation system 300 of FIGS. 3A-3C. In addition, the cannula 310 is depicted as transparent to show the implant or dry implant formulation 360 loaded into the lumen 316 of the cannula 310, as well as to show the distal tip 322 of the pusher rod 320 inserted inside the lumen 316 and contacting the implant or dry implant formulation 360. Therefore, upon advancement of the pusher rod 320 through the lumen 316 and retraction of the cannula 310, implant or dry implant formulation 360 is ejected through the distal opening 318.

Method of Loading a Cannula Pre-Loaded with a Dry Implant Formulation

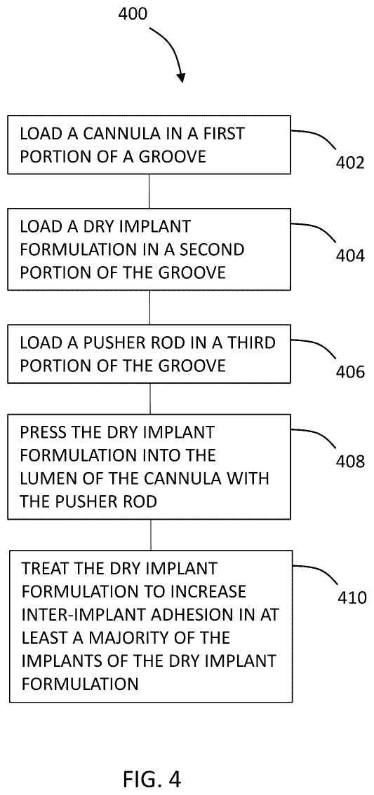

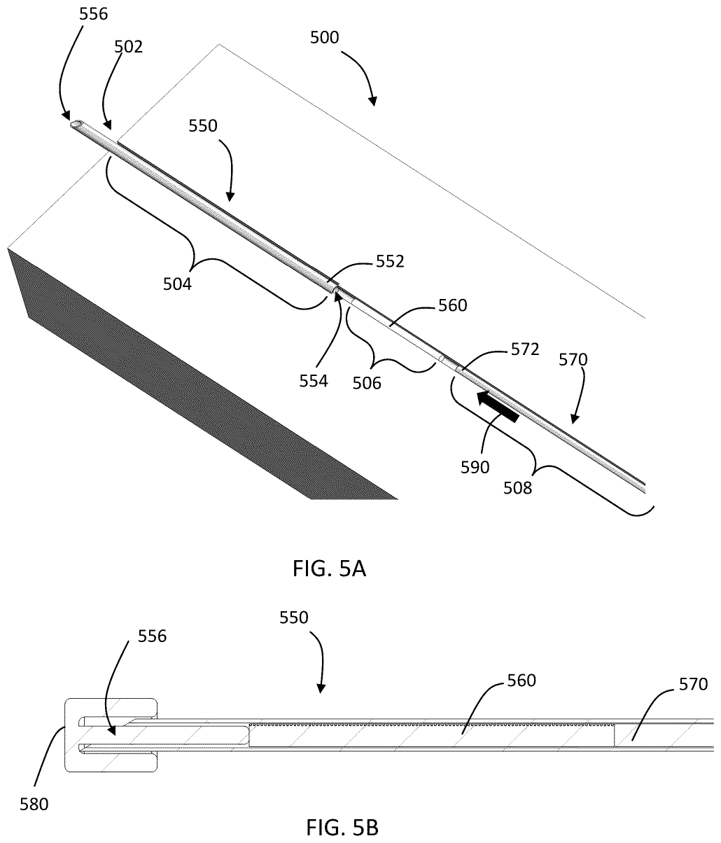

Loading one or more implants, and/or a dry implant formulation, as described herein into an implantation system may be challenging due to the size and characteristics of the implant and/or dry implant formulation as well as the size and configuration of the implantation system (e.g., narrow lumen of a cannula). As such, in some variations, one or more specialized methods and/or loading tools may be utilized to facilitate loading of an implant and/or dry implant formulation into an implantation system. In some variations, the loading process may be performed with a loading block comprising a planar surface, wherein the groove is formed on the planar surface, and is configured to receive a cannula (e.g. a cannula of an implantation system as described herein), a plurality of implants or a dry implant formulation as described herein, and a tamping device or a pusher rod. The loading block may be configured to heat, cool, and/or apply electro static discharge at or near the groove to facilitate implant installation. For example, FIG. 4 provides a flowchart of a method 400 to load an implant or a dry implant formulation, or in the variation provided in FIG. 5 specifically, a dry implant formulation, into a lumen of a cannula of an implantation system. As shown in FIG. 4, a production method 400 may comprise: in a step 402, loading a cannula in a first portion of a groove dimensioned to fit the cannula; in a step 404, loading a dry implant formulation (or one or more implants) in a second portion of the groove near the proximal end of the cannula; and in a step 406, loading a pusher rod in a third portion of the pusher rod so that the loaded dry implantation formulation (or one or more implants) is placed between the proximal end of the cannula and the distal end of the pusher rod.

After the cannula, implant(s) and/or dry implant formulation, and pusher rod are positioned in the groove, the method 400 may further comprise, in a step 408, advancing the pusher rod towards the cannula, thereby pressing the implant(s) and/or dry implant formulation into the lumen of the cannula through the proximal opening of the lumen. The pusher rod may be advanced such that the distal end of the pusher rod enters the lumen of the cannula. In some variations, the pusher rod may be advanced until the implant(s) or a portion of the dry implant formulation is positioned near a distal opening of the cannula.

In some variations, such as those in which a dry implant formulation (which may or may not comprise a binding agent) is used, after the dry implant formulation is positioned within the lumen of the cannula, the method may include, in a step 410, treating the dry implant formulation to increase inter-implant adhesion between at least a majority of the implants of the dry implant formulation. In some variations, the treatment may include heating at least a majority of the implants above respective glass transition temperatures of the polymer or polymers comprised in the drug eluting matrix of the individual implants. The glass transition temperature of an implant is based on the substances comprised therein, for example a bio-erodible polymer comprised in a drug-eluting matrix of the implant. In some variations, the glass transition temperature of an implant may be between about 35 degrees Celsius (degC) and about 45 degC, between about 40 degC and about 45 degC, or between about 37 degC and about 40 degC. In some variations, the treating may include compressing the dry implant formulation in the lumen of the cannula, using, for example, a separate tamping device and/or the pusher rod of the implantation system. In some variations, treating may include both heating and compressing. For example, in some variations, the cannula containing the dry implant formulation may be heated so that at least a majority of the implants of the dry implant formulation are heated above the glass transitions temperatures of their respective polymer(s), and the implants of the dry implant formulation may then be compressed (during or shortly after heating, before the temperature of the majority of the implants fall below the respective glass transition temperatures) by inserting a tamping device into the lumen of the cannula and/or by advancing a pusher rod or tamping device while the cannula is capped (e.g., with a removable cap positioned on the distal end thereof) such that the implants are compressed between the pusher rod or tamping device and the cap. Without being bound by theory, heating above the glass transition temperature, applying compression, or combining heating with compression during or shortly after heating induces the surface of individual implants to be partially deformed against neighboring implants.

FIG. 5A schematically depicts an exemplary method of loading a cannula with one or more implants and/or a dry implant formulation, using a loading block 500 comprising a groove 502 dimensioned to fit a cannula of an implantation system as described herein. In the exemplary variation depicted, a distal portion of the cannula 550 is positioned within a first portion 504 of the groove and implants(s) and/or a dry implant formulation comprising a plurality of microparticle implants is positioned in a second portion of the groove 506 near (e.g., adjacent) a proximal end 602 of the cannula 550. A pusher rod or tamping device 570 is positioned within a third portion 508 of the groove 502, proximal the implant and/or dry implant formulation. In this manner, the implant and/or dry implant formulation 560 is positioned between the proximal end 552 of the cannula and the distal tip 572 of the pusher rod or tamping device 570. The pusher rod or tamping device 570 is then advanced towards the cannula (schematically shown in the direction of block arrow 640), thereby pushing the implant and/or dry implant formulation 560 into the lumen of the cannula 550 through the proximal opening 554 of the lumen of the cannula 550. In other variations, the cannula 550 may be loaded into the loading block 500 in reverse, such that the implant and/or dry implant formulation 560 is loaded in a similar manner through the distal opening 556 of the cannula 560. In some variations, the loading block 500 may be heated so that at least a majority of the microparticle implants in the dry implant formulation is heated to be above the glass transition temperature as described in more detail herein. In some variations, while not depicted in FIG. 5A, a cap may be removably positioned on the end (e.g., distal end) of the cannula 510 during implant and/or dry implant formulation loading. In variations in which compression is with or instead of heating, the pusher rod or tamping device 570 may be advanced until sufficient compression force is applied to the implant and/or dry implant formulation. For example, the pusher rod or tamping device 570 may be advanced so that a distal end of the implant and/or dry implant formulation contacts the cap and the proximal end of the implant and/or dry implant formulation continues to advance distally thereby compressing the implant and/or dry implant formulation. In some variations, the pusher rod of an implantation system may be used in addition to, or instead of, the pusher rod or tamping device 570 to apply a compressive force to the implant and/or dry implant formulation.

FIG. 5B shows a cannula 550 loaded with a dry implant formulation 560, after compression has been applied by the pusher rod or a tamping device 570. The cannula 550 has a cap 580 releasably coupled to a distal opening 556 of the cannula 550. When coupled to the cannula 550, the cap 580 may be positioned over a distal opening 556 of the cannula 550. The cap 580 may further provide protection of the distal end 556 before the cannula is used for an implantation process, e.g. during transport and storage, and the cap 580 may be removed by a user before the implantation process.

### Methods of Treating Conditions of the Eye

Methods of treating a condition of the eye of a subject using the implants or one or more implant formulations (e.g., a dry implant formulation as disclosed herein) are also provided. In general, the methods described herein may comprise implanting at least one drug-eluting implant or an implant formulation(e.g., a dry implant formulation as disclosed herein), including a plurality or a plurality of sets or groups of implants, in the eye of the subject. The methods described herein may involve implantation of one or more implants or implant formulations (e.g., a dry implant formulation) described herein with or without a carrier. When administered with a carrier/vehicle, saline, water, or other non-toxic aqueous media may be used as the carrier/vehicle. One particular advantage of the methods described herein is that the drug-eluting implants are configured to be implanted without the use of a liquid carrier. Implantation of the drug-eluting implants without the use of a liquid carrier may be referred to here as “dry” implantation. Dry implantation limits or eliminates dangerous changes in intraocular pressure that might occur within the eye when a liquid carrier is injected. Additionally, due to at least the size, shape, and structure of the implants as well as the properties of an implant formulations disclosed herein (e.g., a dry implant formulation), a plurality of implants may be implanted closely to one another, and their movement within the eye may be more limited than implants utilizing an aqueous administration, which may assist in avoiding extravasation typically seen with aqueous administration. Other advantages of administration without a carrier are higher drug dose per unit volume injected, and elimination of the need for the drug implantation device to be shipped refrigerated or frozen. The dose may be deployed immediately without extra steps or opportunities for mixing errors. Additionally, dry implantation allows the implants to coalesce in one area, increasing visibility to a physician to determine presence and therefore elution of drug in a particular part of the eye. Further, it allows for the implants to be removed more easily should an adverse event occur. In some embodiments, at least one drug-eluting implant is implanted without a carrier.

### Implantation Locations

In general, a drug may be delivered from at least one drug-eluting implant (optionally as an implant formulation, e.g., a dry implant formulation, as described herein) to a structure of the eye to reduce a symptom of a condition of the eye, as described previously. In some embodiments, the methods may comprise implanting at least one drug-eluting implant or an implant formulation (e.g., a dry implant formulation as disclosed herein) in the anterior chamber, and delivering a drug from the implant to the anterior chamber, and/or to another location in the eye (e.g., posterior chamber, sclera, vitreous, subconjunctival space, Tenon's capsule, sub-Tenon's space) to treat the condition of the eye and/or to reduce one or more symptoms of the condition of the eye. In some embodiments, methods may comprise implanting at least one drug-eluting implant or implant formulation (e.g., a dry implant formulation as disclosed herein) in the vitreous, and delivering a drug from the at least one implant or implant formulation (e.g., a dry implant formulation as disclosed herein) to the vitreous, and/or to another location in the eye (e.g., posterior chamber, sclera, anterior chamber, subconjunctival space) to treat the condition of the eye and/or to reduce one or more symptoms of the condition of the eye. In some embodiments, methods may comprise implanting at least one drug-eluting implant or implant formulation (e.g., a dry implant formulation as disclosed herein) in the subconjunctival space, and delivering a drug from the implant or implant formulation (e.g., a dry implant formulation as disclosed herein) to the subconjunctival space, and/or to another location in the eye (e.g., posterior chamber, sclera, anterior chamber, vitreous, Tenon's capsule, sub-Tenon's space) to treat the condition of the eye and/or to reduce one or more symptoms of the condition of the eye. In some embodiments, methods may comprise implanting at least one drug-eluting implant or implant formulation (e.g., a dry implant formulation as disclosed herein) in the Tenon's capsule or sub-Tenon's space, and delivering a drug from the implant or implant formulation (e.g., a dry implant formulation as disclosed herein) to the Tenon's capsule or sub-Tenon's space, and/or to another location in the eye (e.g., posterior chamber, sclera, anterior chamber, vitreous, subconjunctival space) to treat the condition of the eye and/or to reduce one or more symptoms of the condition of the eye. In some embodiments, methods may comprise implanting at least one drug-eluting implant or implant formulation (e.g., a dry implant formulation as disclosed herein) in the posterior chamber (including, in the sulcus and/or in the remainder of the posterior chamber), and delivering a drug from the at least one drug-eluting implant or implant formulation (e.g., a dry implant formulation as disclosed herein) to the posterior chamber, and/or to another location in the eye (e.g., subconjunctival space, sclera, anterior chamber, vitreous) to treat the condition of the eye and/or to reduce one or more symptoms of the condition of the eye. In some embodiments, the methods described herein may comprise implanting at least one drug-eluting implant or implant formulation (e.g., a dry implant formulation as disclosed herein) fully intramurally, and delivering a drug from the implant(s) or implant formulation to another location (e.g., anterior chamber, posterior chamber, vitreous) of the eye to reduce a symptom of the condition of the eye. Any of the above-mentioned drug-eluting implants or implant formulation (e.g., a dry implant formulation as disclosed herein) and implantation systems are suitable for use with the methods described herein.

### Anesthesia

Prior to administering the drug-eluting implant, the eye may be anesthetized, and one or more antiseptics may be applied to the eye to prepare it for the implantation procedure. Anesthesia may include one or a combination of the following types of anesthesia: topical, subconjunctival, Tenon's capsule, sub-Tenon's space, peribulbar, and retrobulbar. In some instances, an eyelid speculum may be applied to expose the ocular surface and prevent the eyelids from closing. In some instances, it may be advantageous to dilate the pupil. The procedure may also be performed at a slit lamp with the patient seated upright, or it may be performed at a microscope with the patient supine. In some embodiments, the implant may be advanced and/or positioned using loupes, a sit lamp, or a surgical microscope. The procedure may be done in an operating room, although, advantageously, the methods described herein are suitable for being performed at a doctor's office or in a minor procedure room, for example using a slit lamp, under direct visualization, under loupe magnification, or under a microscope. The methods described herein may be performed with or without the use of gonioscopy or microinvasive glaucoma surgery (MIGS)-type implantations. Instead, many of the methods described herein allow for injection into a tissue of the eye, performed at a slit lamp or in an office-based setting by an ophthalmologist or optometrist. While these procedures may be performed in an operating room, there are many advantages to performing them in an office-based setting (e.g., cost savings, convenience for the patients, increased appointment availability and/or access).

### Implantation Process

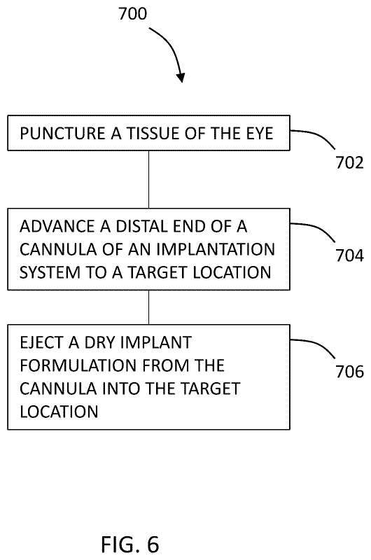

In some variations, a cannula pre-loaded with one or more implants or an implant formulation (e.g., a dry implant formulation as disclosed herein) may be advanced through an external tissue (e.g., sclera, conjunctiva, cornea) to reach the desired or target location or position within the eye. The implant formulation may then be ejected or otherwise released from the implantation system into the target location. In some variations, as shown in FIG. 6, an implantation method 700 may comprise, in a step 702, puncturing a tissue of the eye (e.g., sclera, cornea), optionally with a needle or a cannula of an implantation system, and, in a step 704, advancing a distal end of a cannula of an implantation system carrying one or more implants or an implant formulation (e.g., a dry implant formulation as disclosed herein) in a lumen of the cannula to a desired implant location, such as directly within a mural tissue or within a space within the eye. The desired implant location may include, for example, the posterior chamber, the anterior chamber, the subconjunctival space, the Tenon's capsule, the sub-Tenon's space, or the vitreous. In some variations, the implantation method may comprise, in a step 706, ejecting or otherwise releasing the one or more implants or the implant formulation (e.g., a dry implant formulation as disclosed herein) from the cannula into the target location. In some variations, the ejecting or releasing may comprise operating an actuator of an implantation system, which may result in moving (e.g., advancing) a pusher rod of an implantation system relative to the cannula, retracting the cannula relative to the pusher rod, or a combination of both. In some variations, methods may comprise creating an intramural tunnel or channel separately from, and prior to, advancing the cannula to the target location. In some variations, the tunnel or channel may be created with an instrument such as a needle or a femtosecond laser. In other variations, the channel or tunnel may be created using the implantation system prior to, or during, advancement of the implant or implant formulation to the desired location. In some variations, the implant or implant formulation may be positioned within the mural tissue directly, or within a tunnel or channel. An actuator of an implantation system may then be operated in order to eject or otherwise release the implant or dry implantation formulation into the mural tissue, tunnel or channel, or other naturally occurring space within the eye.

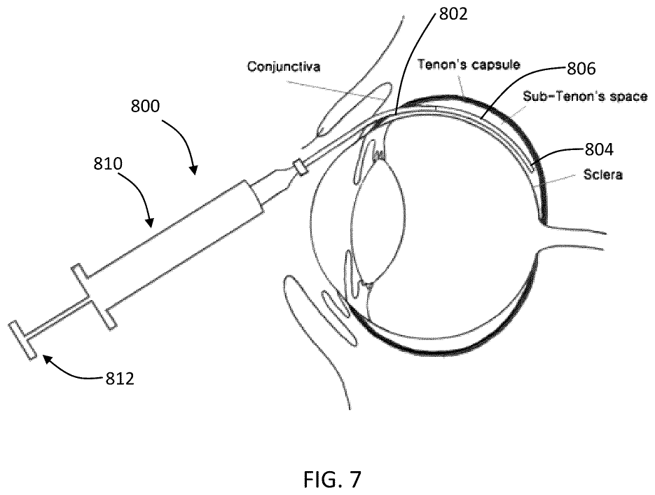

In some variations, the target location may be Sub-Tenon's space. FIG. 7 depicts an exemplary method of implantation into Sub-Tenon's space. In this variation, a distal portion 804 of a cannula 802 of an implantation system 800 may puncture the conjunctiva and the Tenon's capsule. The cannula 802 may be loaded with at least one implant or a dry implant formulation 806, which may be positioned within a lumen of the cannula. The distal end 804 may be advanced into the Sub-Tenon's space. Once the distal end 804 is in the sub-Tenon's space, the actuator on the handle of the implantation system may be operated to eject or otherwise release the implant or dry implant formulation 806 within the cannula 802 into the sub-Tenon's space. In the variation depicted in FIG. 7, the handle of the implantation system is a syringe 810, and the actuator is a plunger 812. It should be appreciated that while depicted with another variation of the implantation system, the method depicted in FIG. 7 may be performed with any of the implantation systems described herein, such as, for example, the implantation system 300 described with respect to FIGS. 3A-3D.

In some variations, a guidewire may be used in the implantation procedure. For example, a guidewire may contact a portion of an implant, and the implant may be advanced from the cannula and/or positioned using the guidewire. After delivering the implant to the target tissue with the guidewire, the implant may be released from the guidewire and/or the guidewire may be withdrawn, leaving the implant in place.

As described above, advancing one or more implants or implant formulations (e.g., a dry implant formulation as disclosed herein) may comprise advancing a portion of an implantation system, e.g., the cannula, through one or more tissues, structures, or membrane of the eye, such as sclera, limbus, or conjunctiva. The method may include advancing the cannula underneath one or more tissues (e.g., conjunctiva, Tenon's capsule). The implant or implant formulation (e.g., a dry implant formulation as disclosed herein) may be disposed within the implantation system. For example, the implant or implant formulation (e.g., a dry implant formulation as disclosed herein) may be disposed within a cannula of the implantation system, and at least a distal end of the cannula may be advanced through a sclera of the eye. The methods described herein may also allow for positioning a drug-eluting implant fully intramurally with at least a portion of the drug-eluting implant in a limbus of the eye. The methods described herein may also allow for delivering a drug from the drug-eluting implant, after placement of the implant, to a target tissue or tissues of the eye (e.g., anterior chamber, subconjunctival space, sub-Tenon's space) to reduce a symptom of the condition of an eye. The target tissue or tissues may be one or more tissues in which the implant resides and/or may be one or more different tissues. The target tissue or tissues may not be in contact with the implant.

The methods described herein are suitable for use with any of the drug-eluting implants including implant formulations (e.g., dry implant formulations as disclosed herein) described herein.

### Visualization of Canmila

After advancing the cannula, wherein the implant(s) or implant formulation (e.g., a dry implant formulation as disclosed herein) is disposed, the cannula may be visualized in a particular portion of the eye or at a particular depth, informing where in the eye the implant will reside once the cannula is retracted. Thus, methods may further comprise visualizing the distal tip of the cannula within a particular tissue, cavity, or structure of the eye (e.g., the anterior chamber) prior to releasing the implant from the implantation system. For instance, the cannula may be visualized within one or more tissues, cavities, or structures of the eye, within which the implant will reside. Additionally, or alternatively, the cannula may be visualized within one or more tissues, cavities, or structures of the eye adjacent to, or within the proximity of, the desired implant location.

Visualizing the cannula through the posterior portion of the eye may assist a user in in properly positioning the implant within the eye, as described above. Accordingly, methods may comprise confirming the distal tip of the cannula is positioned within a desired portion of the eye, such as, but not limited to, within the anterior chamber, posterior chamber, limbus, subconjunctival space, or vitreous of the eye. Once the distal tip of the cannula has been advanced to a desired depth within the eye, methods may further comprise retracting the cannula and/or advancing a pusher, and releasing the drug-eluting implant in the target tissue (e.g., at least partially in the anterior chamber, posterior chamber, limbus, subconjunctival space, or vitreous).

In some variations, a portion of the implantation system (e.g., the cannula) and/or drug-eluting implant may be visualized during advancement and/or positioning using loupes, a slit lamp, a surgical microscope, or any combination thereof. Additionally, or alternatively, a drug-eluting implant may be implanted in a structure of the eye (e.g., suprachoroidal space) gonioscopically.

### Spread of Drug to Locations Beyond Location of Implantation

The drug-eluting implants, including implant formulations (e.g., a dry implant formulation as disclosed herein) may deliver drugs to various locations, irrespective of where in the eye they reside. In any of the methods described herein, a drug-eluting implant (or implants) may deliver a drug to a location or tissue(s) in which the drug-eluting implant resides, and/or to a location or tissue(s) that is different from the location or tissue in which the drug-eluting implant resides. For instance, a drug-eluting implant may reside fully intramurally, within the posterior chamber, within the subconjunctival space, or within the vitreous, but may deliver a drug to the anterior chamber of the eye (or vice versa) via diffusion through the tissue(s). A drug-eluting implant residing fully in the subconjunctival space or sub-Tenon's space, may deliver a drug to one or more of the limbus, sclera, cornea, anterior chamber, ciliary body, trabecular meshwork, choroid, retina, retinal pigment epithelium (RPE), posterior chamber, vitreous, or any other nearby tissue or structure of the eye. A drug-eluting implant residing fully in the vitreous may deliver a drug to one or more of the limbus, sclera, cornea, anterior chamber, posterior chamber, or any other nearby tissue or structure of the eye. A drug-eluting implant residing fully in the sulcus may deliver a drug to one or more of the limbus, sclera, cornea, anterior chamber, vitreous, posterior chamber, or any other nearby tissue or structure of the eye. A drug-eluting implant residing partially in the limbus and partially in the sclera may deliver a drug to one or more of the limbus, sclera, cornea, anterior chamber, or any other tissue or structure of the eye. In certain variations, the drug from the drug-eluting implant may diffuse through one or more aqueous outflow channels. In some embodiments, a plurality of microspheres is implanted in a sulcus of an eye.

### Ocular Conditions

The methods of treating a condition in of an eye of a subject, as described herein, may be useful for treating a number of ocular disorders or conditions. These ocular disorders include, but are not limited to, glaucoma, dry eye, AMD, choroidal diseases, retinal diseases, corneal diseases, iris diseases, uveal diseases, lens diseases, and scleral diseases (e.g., myopia). In some embodiments, the methods described herein may be useful for treating macular edema, vascular occlusions, diabetic retinopathy, retinal degenerations, and retinal dystrophies, iritis, uveitis, vitritis, cataracts, herpes zoster or simplex infection, keratitis, keratoconus or other corneal degenerations, dry eye disease, scleritis, episcleritis, corneal ulcer, astigmatism, hyperopia, presbyopia, cornea ectasia, corneal dystrophies, corneal scars, graft versus host disease, autoimmune ocular diseases, Thygeson's keratitis, post-viral keratitis, herpes simplex, viral keratitis, uveitis, Stevens Johnson Disease, conjunctivitis, blepharitis, postoperative inflammation, postoperative infection prophylaxis, postoperative pain, pingueculum, pingueculitis, pterygium, vernal and atopic keratoconjunctivitis, allergic conjunctivitis, chemical injuries, thermal injuries, chemical injuries, mechanical injuries, retinal vasculitis, retinal dystrophies, neuroretinopathies, autoimmune retinal diseases, autoimmune choroidal diseases, retinal detachment, retinal tears, retinal breaks, ischemic and nonischemic optic neuropathies, tapetoretinal dystrophies, ocular trauma, radiation retinopathy, exudative or nonexudative age related macular degeneration, choroidal neovascularization, retinal neovascularization, retinal vascular occlusive disease, choroidal or retinal inflammation, vitreous opacities (e.g., hemorrhage, floaters, asteroid hyalosis), maculopathies, retinopathies, choroidopathies, retinopathy of prematurity, endophthalmitis, epiretinal membrane hole, macular hole, proliferative vitreoretinopathies, edema (e.g., macular, retinal), ischemia (e.g., macular, retinal), or diabetic retinopathy. In some variations, the methods described herein may result in decreased duration, severity, and/or occurrence of one or more symptoms of any of the aforementioned conditions and/or may result in treatment of any of the aforementioned conditions. The methods described herein, therefore, may utilize a drug-eluting implant that delivers a drug to treat any of these, or other, disorders or conditions. In some embodiments, the dug-eluting implant may deliver a glaucoma drug. In some variations, the methods described herein may utilize a drug-eluting implant located partially in a first part of the eye to treat a disorder in the same part of the eye or another part of the eye.

### Multiple Implantations

The methods described herein may comprise delivering one or more implants, or implantation units of an implant formulation (e.g., a dry implant formulation described herein), to the eye. Multiple implants or implantation units of an implant formulation (e.g., a dry implant formulation as disclosed herein) may be implanted for various reasons or purposes. For example, previously implanted implants or implantation units of an implant formulation (e.g., a dry implant formulation as disclosed herein) may have become either depleted or dissolved. Multiple doses of a drug or multiple different drugs may be implanted during a same sitting at same location or different location, or may be implanted at staggered times. The implants, or implantation units of an implant formulation (e.g., a dry implant formulation as disclosed herein), may be delivered simultaneously or sequentially, and may reside in the eye simultaneously and/or sequentially (e.g., the implants or implantation units of the implant formulation may all be implanted for the same period of time, for different, overlapping periods of time, or for different non-overlapping periods of time). In variations in which a plurality of implants are employed, any number of implants (e.g., one, a plurality, a subset of all implanted, , or implantation units of an implant formulation all implanted) may comprise the same drug, may comprise different drugs with the same mechanism of action for one or more conditions of the eye, or may comprise different drugs with different mechanisms of action for one or more conditions of the eye. The implants, or implantation units of an implant formulation (e.g., a dry implant formulation as disclosed herein) may comprise a drug intended to treat or reduce a symptom of the same condition of the eye, or may comprise a drug intended to treat or reduce a symptom of different conditions of the eye. Moreover, the implants , or implantation units of an implant formulation (e.g., a dry implant formulation as disclosed herein) may be positioned in the same general location in the eye or in different areas in the eye. It should be appreciated that while the implants are described as comprising a drug, this may include combinations of drugs. In other words, multiple implants , or implantation units of an implant formulation (e.g., a dry implant formulation as disclosed herein)may be delivered sequentially during the same procedure or may be delivered simultaneously. In variations in which the implants or implantation units of the implant formulation are delivered sequentially, the implants or implantation units of the implant formulation may be advanced to a target implant location (e.g., sulcus, posterior chamber, anterior chamber vitreous, sub-Tenon's space, subconjunctival space) together (e.g., while contained within a common implantation system), or the implants or implantation units of the implant formulation may be advanced to the target implant location separately (e.g., positioned one at a time in a common implantation system, using different implantation systems).

For example, methods may comprise positioning a first implant or implants (which may be a first subset of implants or a first implant formulation) comprising a first drug or combination of drugs intended to treat or reduce a symptom of a first condition in a first location in the eye and positioning a second implant or implants (which may be a second subset of implants or a second implant formulation) comprising a second drug or combination of drugs intended to treat or reduce a second condition in a second location in the eye. In some variations, the first drug or combination drugs and the second drug or combination of drugs may be the same drug or combination of drugs, the first condition and the second condition may be the same condition, the first implantation location and the second implantation location may be the same location, and/or the first drug or combination of drugs and the second drug or combination of drugs may utilize the same mechanism of action. In other variations, the first drug or combination of drugs and the second drug or combination of drugs may be different drugs or different combinations of drugs, the first condition and the second condition may be different conditions, the first implantation location and the second implantation location may be different locations, and/or the first and second drugs or combination of drugs may utilize different mechanisms of action. Thus, in some variations, the first and second drugs or combinations of drugs may be different drugs or combinations that utilize different mechanisms of action, but the first and second locations may be the same location and the first and second conditions may be the same condition. In another example, the first and second drugs or combinations of drugs may be different drugs or different combinations of drugs that utilize different mechanisms of action and the first and second locations may be different locations, but the first and second conditions of the eye may be the same. It should be appreciated that any combination of drugs, mechanisms of action, locations, and conditions of the eye described herein may be used in combination when utilizing methods comprising use of multiple drug-eluting implants. In any of the embodiments described herein, a drug or combination of drugs may be delivered to one or more locations in different amounts (e.g., a first amount of a drug or drug combination in a first location and a second amount of the drug or the drug combination in a second location).

For some subjects, it may be advantageous to deliver multiple implants or implant formulations (e.g., a dry implant formulation as disclosed herein) configured to deliver different drugs or to deliver drugs that utilize different mechanisms of action as this may allow for a more comprehensive treatment. For example, methods may comprise positioning a first implant or implants (which may be a first subset of implants in an implant formulation or a first implant formulation) comprising a first drug with a first mechanism of action in the sulcus and positioning a second implant or implants (which may be a second subset of implants in an implant formulation or a second implant formulation)comprising a second drug with a second mechanism of action at least partially in the anterior chamber or posterior chamber. In some variations, the first mechanism of action may be suppression of production of aqueous humor and the second mechanism of action may be increasing the drainage of aqueous humor using one or more of the trabeculocanicular pathway and the uveoscleral pathway. In other variations, both the first mechanism of action and the second mechanism of action may be suppression of aqueous humor or increasing drainage of aqueous humor using one or more of the trabeculocanicular pathway and the uveoscleral pathway. In some variations, the first and second mechanisms of action may be increasing drainage of aqueous humor, however, the first mechanism of action may be increasing drainage through the trabeculocanicular pathway and the second mechanism of action may be increasing drainage through the uveoscleral pathway. In some variations, the first implant may comprise a drug for treating or reducing one or more symptoms of glaucoma (e.g., by suppression of aqueous humor, increasing drainage of aqueous humor using the trabeculocanicular pathway, increasing drainage of aqueous humor using the uveoscleral pathway) or a condition of the retina, lens, cornea, uvea, vitreous, iris, ciliary body, sclera, or ocular surface, and the second implant may comprise a drug for treating or reducing one or more symptoms of glaucoma or a condition of the retina, lens, cornea, uvea, vitreous, iris, ciliary body, sclera, or ocular surface. For example the first implant or implants (which may be a first subset of implants in an implant formulation or a first implant formulation)and the second implant or implants (which may be a second subset of implants in an implant formulation or a second implant formulation) may each comprise a drug for treating or reducing one or more symptoms of glaucoma, and the drug may be the same drug (or combination of drugs) or a different drug (or different combination of drugs). In some variations, for reducing ocular pressure to, e.g., treat glaucoma, the first mechanism of action may be blocking of beta-2-adrenergic receptors (e.g. using a timolol) and the second mechanism of action may be inhibiting carbonic anhydrase (e.g. using a brinzolamide or a dorzolamide). In other variations, for reducing ocular pressure to, e.g., treat glaucoma, the first mechanism of action may be blocking of beta-2-adrenergic receptors (e.g. using a timolol) and the second mechanism of action may be administration of a prostaglandin analog or a prostamide analog (e.g. using a latanoprost or bimatoprost). In other variations, for reducing ocular pressure to, e.g., treat glaucoma, the first mechanism of action may be blocking of beta-2-adrenergic receptors (e.g. using a timolol) and the second mechanism of action may be inhibition of a rho-kinase (e.g. using a ripasudil or netarsudil.). In other variations, for reducing ocular pressure to, e.g., treat glaucoma, the first mechanism of action may be administration of a prostaglandin analog or a prostamide analog (e.g. using a latanoprost or bimatoprost), and the second mechanism of action may be inhibition of a rho-kinase (e.g. using a ripasudil or netarsudil.). In another example, the first implant or implants (which may be a first subset of implants in an implant formulation or a first implant formulation)may comprise a drug for treating or reducing one or more symptoms of glaucoma and the second implant or implants (which may be a second subset of implants in an implant formulation or a second implant formulation) may comprise a drug for treating or reducing one or more symptoms of retinal disease. In some variations, the first implant or implants (which may be a first subset of implants in an implant formulation or a first implant formulation)and the second implant or implants (which may be a second subset of implants in an implant formulation or a second implant formulation)may each comprise a drug for treating or reducing one or more symptoms of AMD, and the drug may be the same drug (or combination of drugs) or a different drug (or combination of drugs). In another example, the first implant or implants (which may be a first subset of implants in an implant formulation or a first implant formulation)may comprise a drug for treating or reducing one or more symptoms of glaucoma and the second implant or implants (which may be a second subset of implants in an implant formulation or a second implant formulation)may comprise a drug for treating or reducing one or more symptoms of AMD. In another example, the first implant or implants (which may be a first subset of implants in an implant formulation or a first implant formulation) may comprise a drug for treating or reducing one or more symptoms of glaucoma and the second implant or implants (which may be a second subset of implants in an implant formulation or a second implant formulation) may comprise a drug for treating or reducing one or more symptoms of dry eye disease.

It should be understood that a first implant or implants (which may be a first subset of implants or a first implant formulation), may be advanced to a first location, and then a second implant or implants (which may be a second subset of implants or a second implant formulation) may be advanced subsequently to a second location. In some variations, a portion of a single subset of implants in an implantation system could be advanced into a first location, and then a subsequent portion or portions could be advanced into a second location (or third, fourth, etc.) from the same implantation system. In some embodiments, a first implant or first subset of implants maybe advanced to a first location in the eye from an implantation system, and the same implantation system may be recharged with an additional implant or subset to one or more additional locations in the eye.

### Exemplary Embodiments

Embodiment I-1. A drug-eluting implant for treating a condition of an eye of a subject comprising a bio-erodible microsphere, wherein the drug-eluting implant is configured to be implanted in an eye of a subject without a carrier.

Embodiment I-2. The drug-eluting implant of embodiment I-1, wherein the bio-erodible microsphere comprises a drug-eluting matrix.

Embodiment I-3. The drug-eluting implant of embodiment I-2, wherein the drug-eluting matrix comprises a bio-erodible polymer and a drug.

Embodiment I-4. The drug-eluting implant of any one of embodiments I-1 to I-3, wherein the bio-erodible microsphere comprises poly(lactic-co-glycolic acid) (PLGA), poly(lactic acid) (PLA), poly-epsilon-caprolactone (PCA), or poly(glycolic acid) (PGA).

Embodiment I-5. The drug-eluting implant of any one of embodiments I-1 to I-4, wherein the bio-erodible microsphere comprises a mixture of two bio-erodible polymers.

Embodiment I-6. The drug-eluting implant of any one of embodiments I-1 to I-5, wherein the bio-erodible microsphere has a diameter of between about 1 μm and about 500 μm.

Embodiment I-7. The drug-eluting implant of any one of embodiments I-1 to I-6, wherein the bio-erodible microsphere has a diameter of between about 10 μm and about 100 μm.

Embodiment I-8. The drug-eluting implant of any one of embodiments I-1 to I-7, wherein the condition of the eye is glaucoma and/or dry eye disease.

Embodiment I-9. The drug-eluting implant of any one of embodiments I-1 to I-8, wherein the drug-eluting implant is configured to be positioned in one or more of a sulcus, a posterior chamber, an anterior chamber, a vitreous, and a subconjunctival space of an eye.

Embodiment I-10. The drug-eluting implant of any one of embodiments I-3 to I-9, wherein the drug is a prostaglandin or a prostaglandin analog.

Embodiment I-11. The drug-eluting implant of any one of embodiments I-3 to I-10, wherein the drug is latanoprost, bimatoprost, travoprost, nerve growth factor, anti-VEGF antibody, or cyclosporine.

Embodiment I-12. The drug-eluting implant of any one of embodiments I-3 to I-11, wherein the bio-erodible microsphere comprises between about 1 μg and about 500 μg of the drug.

Embodiment I-13. The drug-eluting implant of any one of embodiments I-3 to I-12, wherein the bio-erodible microsphere comprises between about 5 μg and about 100 μg of the drug.

Embodiment I-14. The drug-eluting implant of any one of embodiments I-3 to I-13, wherein the drug is delivered to the eye at a rate of between about 1 ng/day and about 3000 ng/day.

Embodiment I-15. The drug-eluting implant of any one of embodiments I-3 to I-14, wherein the drug is delivered to the eye at a rate of between about 5 ng/day and about 2000 ng/day.

Embodiment I-16. The drug-eluting implant of any one of embodiments I-3 to I-15, wherein the drug is delivered to the eye over a period of time, wherein the period of time is at least 1 month, at least 4 months, at least 6 months, at least 8 months, at least 1 year, at least 2 years, or at least 3 years.

Embodiment I-17. The drug-eluting implant of any one of embodiments I-1 to I-16, wherein the microsphere comprises an imaging agent.

Embodiment I-18. The drug-eluting implant of embodiment I-17, wherein the imaging agent is one or more of a dye, a radiolabel, and a fluorescent marker.

Embodiment I-19. The drug-eluting implant of embodiments I-17 to I-18, wherein the imaging agent is fluorescein.

Embodiment I-20. The drug-eluting implant of any one of embodiments I-17 to I-19, wherein the microsphere comprises a drug and an imaging agent, and wherein the microsphere is configured to deliver the drug and the imaging agent into the eye at the same rate.

Embodiment I-21. A method for treating a condition of an eye of a subject, the method comprising:
implanting at least one drug-eluting implant in the eye of the subject, wherein the at least one implant is implanted without a carrier; and
wherein a drug is delivered from the at least one drug-eluting implant to the eye to reduce a symptom of the condition of the eye.

Embodiment I-22. The method of embodiment I-21, wherein the condition of the eye is glaucoma and/or dry eye disease.

Embodiment I-23. The method of embodiment I-21 or I-22, wherein the at least one drug-eluting implant is delivered from the at least one drug-eluting implant to a sulcus, a posterior chamber, an anterior chamber, a vitreous, and a subconjunctival space of the eye.

Embodiment I-24. The method of any one of embodiments I-21 to I-23, further comprising advancing the at least one drug-eluting implant to an implantation site in the eye in an implantation system.

Embodiment I-25. The method of embodiment I-24, wherein the advancing step comprises pushing the at least one drug-eluting implant with a pusher rod.

Embodiment I-26. The method of embodiment I-24 or I-25, wherein the implantation system comprises a needle or cannula, and the method further comprises puncturing a tissue of the eye with the needle or cannula.

Embodiment I-27. The method of any one of embodiments I-24 to I-26, wherein the method further comprises operating an actuator of the implantation system to release the at least one drug-eluting implant from the implantation system.

Embodiment I-28. The method of embodiment I-27, wherein more than one drug-eluting implant is released from the implantation system.

Embodiment I-29. The method of embodiment I-28, wherein operating the actuator advances the pusher rod.

Embodiment I-30. The method of any one of embodiments I-27 to I-29, wherein the actuator comprises one or more of a button, a knob, a slider, a lever, and a wheel.

Embodiment I-31. The method of any one of embodiments I-21 to I-30, wherein the at least one drug-eluting implant is advanced and/or positioned using one or more of a loop, a slit lamp, and a surgical microscope.

Embodiment I-32. The method of any one of embodiments I-21 to I-31, wherein the at least one drug-eluting implant delivers a glaucoma drug and/or a dry eye disease drug.

Embodiment I-33. The method of any one of embodiments I-21 to I-32, wherein the at least one drug-eluting implant delivers a prostaglandin or a prostaglandin analog.

Embodiment I-34. The method of any one of embodiments I-21 to I-33, wherein the at least one drug-eluting implant delivers latanoprost, bimatoprost, travoprost, nerve growth factor, anti-VEGF antibody or cyclosporine.

Embodiment I-34. The method of any one of embodiments I-21 to I-34, wherein the at least one drug-eluting implant comprises a first drug-eluting implant, and the method further comprises implanting a second drug-eluting implant in the eye.

Embodiment I-36. The method of embodiment I-35, wherein the first drug-eluting implant comprises a first drug with a first mechanism of action, and the second drug-eluting implant comprises a second drug with a second mechanism of action.

Embodiment I-37. The method of embodiment I-36, wherein the first mechanism of action and the second mechanism of action are the same.

Embodiment I-38. The method of embodiment I-36, wherein the first mechanism of action is different from the second mechanism of action.

Embodiment I-39. The method of any one of embodiments I-34 to I-38, wherein the first drug-eluting implant is implanted in a first site in the eye, and the second drug-eluting implant is implanted in a second site in the eye.

Embodiment I-40. The method of any one of embodiments I-34 to I-38, wherein the first drug-eluting implant and the second drug-eluting implant are implanted in the same site in the eye.

Embodiment I-41. The method of any one of embodiments I-21 to I-40, wherein the at least one drug-eluting implant comprises a plurality of microspheres, and wherein the plurality of microspheres is implanted in a sulcus of the eye.

Embodiment II-1. A dry implant formulation for treating a condition of an eye of a subject comprising a plurality of drug-eluting microparticle implants, wherein the dry implant formulation is configured to be implanted in an eye of a subject without a carrier.

Embodiment II-2. The dry implant formulation of embodiment II-1, wherein each of the plurality of microparticle implants comprise a drug-eluting matrix.

Embodiment II-3. The dry implant formulation of embodiment II-2, wherein the drug-eluting matrix comprises a bio-erodible polymer and a drug.

Embodiment II-4. The drug-eluting implant of any one of embodiments II-1 to II-3, wherein the microparticle implants comprises poly(lactic-co-glycolic acid) (PLGA), poly(lactic acid) (PLA), poly-epsilon-caprolactone (PCA), or poly(glycolic acid) (PGA).

Embodiment II-5. The drug-eluting implant of any one of embodiments II-1 to II-4, wherein the microparticle implants comprises a mixture of two bio-erodible polymers.

Embodiment II-6. The dry implant formulation of any one of embodiments II-1 to II-5, wherein each of the plurality of the microparticle implants has a diameter of between about 1 um and about 500 μm.

Embodiment II-7. The dry implant formulation of any one of embodiments II-1 to II-6, wherein each of the plurality of the microparticle implants has a maximum linear dimension of between about 10 μm and about 100 μm.

Embodiment II-8. The dry implant formulation of any one of embodiments II-1 to II-7, wherein the condition of the eye is glaucoma and/or dry eye disease.

Embodiment II-9. The dry implant formulation of any one of embodiments II-1 to II-8, wherein the dry implant formulation is configured to be positioned in one or more of a sulcus, a posterior chamber, an anterior chamber, a vitreous, a sub-Tenon's space, and a subconjunctival space of an eye.

Embodiment II-10. The dry implant formulation of any one of embodiments II-3 to II-9, wherein the drug is a prostaglandin or a prostaglandin analog.

Embodiment II-11. The dry implant formulation of any one of embodiments II-3 to II-10, wherein the drug is latanoprost, bimatoprost, travoprost, a beta-adrenergic blocker, a carbonic anhydrase inhibitor, a nerve growth factor, an anti-VEGF antibody, or cyclosporine.

Embodiment II-12. The dry implant formulation of any one of embodiments II-3 to II-11, wherein the plurality of the microparticle implants comprises in total between about 5 μg and about 15 mg of the drug.

Embodiment II-13. The dry implant formulation of any one of embodiments II-3 to II-12, wherein the plurality of the microparticle implants comprises in total between about 10 μg and about 10 mg of the drug.

Embodiment II-14. The dry implant formulation of any one of embodiments II-3 to II-13, wherein the plurality of the microparticle implants in total is configured to deliver the drug to the eye at a rate of between about 1 ng/day and about 50 μg /day.

Embodiment II-15. The dry implant formulation of any one of embodiments II-3 to II-14, wherein the plurality of the microparticle implants in total is configured to deliver the drug to the eye at a rate of between about 5 ng/day and about 2000 ng/day.

Embodiment II-16. The dry implant formulation of any one of embodiments II-3 to II-15, wherein dry implant formulation is configured to deliver the drug to the eye over a period of time, wherein the period of time is at least 1 month, at least 4 months, at least 6 months, at least 8 months, at least 1 year, at least 2 years, or at least 3 years.

Embodiment II-17. The dry implant formulation of any one of embodiments II-1 to II-16, wherein at least a subset of the plurality of the microparticle implants comprises an imaging agent.

Embodiment II-18. The dry implant formulation of embodiment II-17, wherein the imaging agent is one or more of a dye, a radiolabel, and a fluorescent marker.

Embodiment II-19. The dry implant formulation of embodiments II-17 to II-18, wherein the imaging agent is fluorescein.

Embodiment II-20. The dry implant formulation of any one of embodiments II-17 to II-19, wherein each of the plurality of the microparticle implants comprises a drug and an imaging agent, and wherein each of the plurality of the microparticle implants is configured to deliver the drug and the imaging agent into the eye at the same rate.

Embodiment II-21. The dry implant formulation of any one of embodiments II-1 to II-20, wherein the dry implant formulation comprises a first microparticle implant comprising a first drug and a second microparticle implant comprising a second, different drug.

Embodiment II-22. The dry implant formulation of embodiment II-21, wherein the first drug is a beta-adrenergic blocker.

Embodiment II-23. The dry implant formulation of embodiment II-22, wherein the beta-adrenergic blocker is a timolol.

Embodiment II-24. The dry implant formulation of embodiment II-23, wherein the timolol is timolol maleate or timolol hemihydrate.

Embodiment II-25. The dry implant formulation of any one of embodiments II-21 to II-24, wherein the second drug is a carbonic anhydrase inhibitor.

Embodiment II-26. The dry implant formulation of embodiment II-25, wherein the carbonic anhydrase inhibitor is a dorzolamide or a brinzolamide.

Embodiment II-27. The dry implant formulation of embodiment II-26, wherein the dorzolamide is dorzolamide hydrochloride or dorzolamide base.

Embodiment II-28. The dry implant formulation of embodiments II-22 to II-24, wherein the second drug is a prostaglandin analog or a prostamide analog.

Embodiment II-29. The dry implant formulation of embodiment II-28, wherein the prostaglandin analog is latanoprost, travoprost, tafluprost, or unoprostone, and the prostamide analog is bimatoprost.

Embodiment II-30. The dry implant formulation of embodiment II-28, wherein the second drug is the prostaglandin analog, and the prostaglandin analog is latanoprost.

Embodiment II-31. The dry implant formulation of embodiments II-22 to II-24, wherein the second drug is a rho-kinase inhibitor.

Embodiment II-32. The dry implant formulation of embodiment II-31, wherein the rho-kinase inhibitor is ripasudil or netarsudil.

Embodiment II-33. The dry implant formulation of embodiment II-21, wherein the first drug is a prostaglandin analog or a prostamide analog.

Embodiment II-34. The dry implant formulation of embodiment II-33, wherein the prostaglandin analog is latanoprost, travoprost, tafluprost, or unoprostone, and the prostamide analog is bimatoprost.

Embodiment II-35. The dry implant formulation of embodiment II-34, wherein the first drug is a prostaglandin analog, and the prostaglandin analog is latanoprost.

Embodiment II-36. The dry implant formulation of any one of embodiments II-32 to II-35, wherein the second drug is a rho-kinase inhibitor.

Embodiment II-37. The dry implant formulation of embodiment II-36, wherein the rho-kinase inhibitor is ripasudil or netarsudil.

Embodiment II-38. A dry implant formulation for treating glaucoma of an eye of a subject comprising a first microparticle implant comprising a timolol and a second microparticle implant comprising a dorzolamide or a brinzolamide, wherein the dry implant formulation is configured to be implanted in sub-Tenon's space in the eye of the subject without a carrier.

Embodiment II-39. The dry implant formulation of any one of embodiments II-1 to II-38, wherein each of the plurality of microparticle implants is a microsphere.

Embodiment II-40. The dry implant formulation of any one of embodiments II-1 to II-39, wherein each of the plurality of microparticle implants comprise a bio-erodible polymer, and at least a majority of the plurality of microspheres have been heated above the glass transition temperature of the bio-erodible polymer.

Embodiment II-41. The dry implant formulation of any one of embodiments II-1 to II-40, further comprising a binding agent.

Embodiment II-42. The dry implant formulation of embodiment II-41, wherein the binding agent is selected from the group consisting of: a sugar, a gelatin, a collagen, polyethylene glycol (PEG), a starch, a cellulose, an alginate, and a chitosan.

Embodiment II-43. The dry implant formulation of embodiment II-42, wherein the binding agent is sugar, and the sugar is glucose, sucrose, lactose, or fructose.

Embodiment II-44. The drug-eluting implant of any one of embodiments II-1 to II-42, wherein the dry implant formulation is malleable, and does not readily dissociate into individual implants.

Embodiment II-45. A system for treating a condition of an eye of a subject, the system comprising: a cannula comprising a lumen and a distal tip configured for insertion into a portion of the eye; and a dry implant formulation comprising a plurality of drug-eluting, microparticle implants positioned within the lumen of the cannula without a carrier.

Embodiment II-46. The system of embodiment II-44, wherein the dry implant formulation is malleable and does not readily dissociate into individual implants.

Embodiment II-47. The system of embodiment II-45 or II-46, wherein the distal tip of the cannula has a beveled edge.

Embodiment II-48. The system of embodiment II-45 or II-46, wherein the cannula comprises a proximal portion and a distal portion terminating at a distal tip, wherein the distal tip is tapered and has a smaller external diameter than the proximal portion.

Embodiment II-49. The system of any one of embodiments II-45 to II-48, wherein the cannula is transparent.

Embodiment II-50. The system of any one of embodiments II-45 to II-49 further comprising a pusher rod slidably positioned within the lumen, wherein a distal tip of the pusher rod is in contact with at least a portion of dry implant formulation.

Embodiment II-51. The system of embodiment II-50, further comprising a handle connected to the cannula, wherein the handle is sized and shaped to comfortably hold and manipulate the cannula

Embodiment II-52. The system of embodiment II-51, wherein the handle comprises an actuator is operatively coupled to the pusher rod, the cannula, or both, wherein engaging the actuator moves the pusher rod, the cannula, or both.

Embodiment II-53. The system of embodiment II-52, wherein the actuator is a wheel configured to be rotated by a user and the handle comprises a drive assembly that translates rotational movement of the wheel into a linear motion of the pusher rod.

Embodiment II-54. The system of embodiment II-53, wherein the drive assembly comprises: a first pinion gear coaxially or tangentially attached to the wheel; an idler gear that engages with the first pinion gear; and a first elongate member connected to the pusher rod , wherein the first elongate member engages with the idler gear to translate a rotational movement of the wheel into a first linear movement of the first elongate member.

Embodiment II-55. The system of embodiment II-54, wherein the first elongate member is a first linear gear.

Embodiment II-56. The system of embodiment II-53, wherein the actuator is operatively coupled to the cannula, and is operable to move the cannula with respect to the handle and the pusher rod, thereby retracting the distal opening of the cannula towards the tip of the pusher rod.

Embodiment II-57. The system of embodiment II-56, wherein the actuator is a wheel configured to be rotated by a user and the handle comprises a drive assembly that translates rotational movement of the wheel into a linear motion of the cannula.

Embodiment II-58. The system of embodiment II-57, wherein the drive assembly comprises: a second pinion gear coaxially or tangentially attached to the wheel; and a second elongate member connected to the cannula, that engages with the pinion gear, to translate a rotational movement of the wheel into a second linear movement of the second elongate member.

Embodiment II-59. The system of embodiment II-58, wherein the second elongate member is a second linear gear.

Embodiment II-60. The system of embodiment II-53, wherein the actuator is operatively coupled to the pusher rod, and is operable to: move the pusher rod with respect to the handle and the cannula, thereby advancing the tip of the pusher rod towards the distal opening of the cannula; and move the cannula with respect to the handle and the pusher rod, thereby retracting the distal opening of the cannula towards the tip of the pusher rod.

Embodiment II-61. The system of embodiment II-60, wherein the actuator is a wheel configured to be rotated by a user and the cannular holder comprises a drive assembly that translates rotational movement of the wheel into a linear motion of the pusher rod and an opposite linear motion of the cannula.

Embodiment II-62. The system of embodiment II-61, wherein the drive assembly comprises: a first pinion gear coaxially or tangentially attached to the wheel; an idler gear that engages with the first pinion gear; a first elongate member connected to the pusher rod, that engages with the idler gear, to translate a rotational movement of the wheel into a first linear movement of the first elongate member; a second pinion gear coaxially or tangentially attached to the wheel; and a second elongate member connected to the cannula, that engages with the pinion gear, to translate a rotational movement of the wheel into a second linear movement of the second elongate member,

wherein the first linear movement and the second linear movement are in opposite directions.

Embodiment II-63. A method of forming a system for treating a condition of an eye of a subject, the method comprising: inserting a dry implant formulation comprising a plurality of drug-eluting microparticle implants into a lumen of a cannula, wherein the cannula is configured for insertion into a target location in an eye, and wherein the dry implant formulation does not comprise a carrier; heating and/or compressing the dry implant formulation, thereby increasing adhesion among the plurality of microparticle implants.

Embodiment II-64. The method of embodiment II-63, comprising heating the cannula, wherein each of the plurality of microparticle implants comprise a bio-erodible polymer, and at least a majority of the plurality of microparticle implants is heated to or above the glass transition temperature of the bio-erodible polymer, thereby increasing adhesion of at least a portion of the microparticle implants to one another.

Embodiment II-65. The method of embodiment II-64, comprising compressing the dry implant formulation while the majority of the plurality of microparticle implants is heated above the glass transition temperature of the bio-erodible polymer.

Embodiment II-66. The method of any one of embodiments II-63 to II-65, wherein the dry implant formulation comprises a binding agent.

Embodiment II-67. The method of embodiment II-66, wherein the binding agent is selected from the group consisting of: a sugar, a gelatin, a collagen, polyethylene glycol (PEG), a starch, a cellulose, an alginate, and a chitosan.

Embodiment II-68. The method of embodiment II-67, wherein the binding agent is sugar, and the sugar is glucose, sucrose, lactose, or fructose

Embodiment II-69. The method of embodiment II-68, wherein the dry implant formulation is compressed, thereby increasing adhesion between the plurality of microparticle implants and the binding agent.

Embodiment II-70. A method for treating a condition of an eye of a subject, the method comprising: implanting at least one drug-eluting implant in the eye of the subject, wherein the at least one implant is implanted without a carrier; and wherein a drug is delivered from the at least one drug-eluting implant to the eye to reduce a symptom of the condition of the eye.

Embodiment II-71. The method of embodiment II-70, wherein the condition of the eye is glaucoma and/or dry eye disease.

Embodiment II-72. The method of embodiment II-70 or II-71, wherein the at least one drug-eluting implant is delivered from the at least one drug-eluting implant to a sulcus, a posterior chamber, an anterior chamber, a vitreous, a sub-Tenon's space, and a subconjunctival space of the eye.

Embodiment II-73. The method of any one of embodiments II-70 to II-72, further comprising advancing the at least one drug-eluting implant to an implantation site in the eye in an implantation system.

Embodiment II-74. The method of embodiment II-73, wherein the advancing step comprises pushing the at least one drug-eluting implant with a pusher rod.

Embodiment II-75. The method of embodiment II-73, wherein the implantation system comprises a cannula, the at least one implant is housed in the cannula, and the method further comprises puncturing a tissue of the eye with the cannula.

Embodiment II-76. The method of embodiment II-74 or II-75, wherein the method further comprises operating an actuator of the implantation system to release the at least one drug-eluting implant from the implantation system.

Embodiment II-77. The method of any one of embodiments II-74 to II-76, wherein more than one drug-eluting implant is released from the implantation system.

Embodiment II-78. The method of any one of embodiments II-74 to II-77, wherein operating the actuator advances the pusher rod.

Embodiment II-79. The method of any one of embodiments II-75 to II-78, wherein the actuator comprises one or more of a button, a knob, a slider, a lever, and a wheel.

Embodiment II-80. The method of any one of embodiments II-70 to II-79, wherein the at least one drug-eluting implant is advanced and/or positioned using one or more of a loop, a slit lamp, and a surgical microscope.

Embodiment II-81. The method of any one of embodiments II-70 to II-80, wherein the at least one drug-eluting implant delivers a glaucoma drug and/or a dry eye disease drug.

Embodiment II-82. The method of any one of embodiments II-70 to II-81, wherein the at least one drug-eluting implant delivers a prostaglandin or a prostaglandin analog.

Embodiment II-83. The method of any one of embodiments II-70 to II-82, wherein the at least one drug-eluting implant delivers a beta-adrenergic blocker; a carbonic anhydrase inhibitor, a prostaglandin analog, an immunosuppressant, a rho-kinase inhibitor, or a combination thereof.

Embodiment II-84. The method of any one of embodiments II-70 to II-83, wherein the at least one drug-eluting implant comprises a first drug-eluting implant, and the method further comprises implanting a second drug-eluting implant in the eye.

Embodiment II-85. The method of embodiment II-84, wherein the first drug-eluting implant comprises a first drug with a first mechanism of action, and the second drug-eluting implant comprises a second drug with a second mechanism of action.

Embodiment II-86. The method of embodiment II-85, wherein the first mechanism of action and the second mechanism of action are the same.

Embodiment II-87. The method of embodiment II-86, wherein the first mechanism of action is different from the second mechanism of action.

Embodiment II-88. The method of any one of embodiments II-84 to II-87, wherein the first drug-eluting implant is implanted in a first site in the eye, and the second drug-eluting implant is implanted in a second site in the eye.

Embodiment II-89. The method of any one of embodiments II-84 to II-87, wherein the first drug-eluting implant and the second drug-eluting implant are implanted in the same site in the eye.

Embodiment II-90. The method of any one of embodiments II-70 to II-89, wherein the at least one drug-eluting implant is a drug-eluting microparticle implant, and is implanted as part of a dry implant formulation comprising a plurality of drug-eluting microparticle implants.

Embodiment II-91. A method for treating a condition of an eye of a subject, the method comprising: advancing a distal portion of a cannula of an implantation system towards a target location in the eye, wherein the cannula comprises a lumen and a distal tip configured to puncture tissue and wherein the cannula houses a dry implant formulation comprising a plurality of drug-eluting microparticle implants without a carrier; actuating the implantation system so that the dry implant formulation is released from the distal portion of the cannula into the target location.

Embodiment II-92. The method of embodiment II-91, wherein each of the plurality of microparticle implants comprises a drug selected from the group consisting of a beta-adrenergic blocker; a carbonic anhydrase inhibitor, a prostaglandin analog, an immunosuppressant, a rho-kinase inhibitor, or a combination thereof

Embodiment II-93. The method of embodiment II-91 or II-92, wherein the target location in the eye is a sulcus, a posterior chamber, an anterior chamber, a vitreous, a sub-Tenon's space, or a subconjunctival space.

Embodiment II-94. The method of any one of embodiments II-91 to II-93, wherein the dry implant formulation , after being released into the target location, deliver the drug to the target location at a rate of between about 1 ng/day and about 50 μg/day.

Embodiment II-95. The method of any one of embodiments II-91 to II-93, wherein the dry implant formulation , after being released into the target location, deliver the drug to the eye at a rate of between about 5 ng/day and about 2000 ng/day.

Embodiment II-96. The method of any one of embodiments II-91 to II-95, wherein dry implant formulation, after being released into the target location, deliver the drug to the eye over a period of time, wherein the period of time is at least 1 month, at least 4 months, at least 6 months, at least 8 months, at least 1 year, at least 2 years, or at least 3 years.

Embodiment II-97. The method of any one of embodiments II-91 to II-96, wherein the dry implant formulation comprises a first subset of microparticle implants comprising a first drug and a second subset of microparticle implants comprising a second, different drug.

Embodiment II-98. The method of embodiment II-96, wherein the condition of the eye is glaucoma or elevated ocular pressure.

Embodiment II-99. The method of embodiment II-97 or II-98, wherein the first drug is a beta-adrenergic blocker.

Embodiment II-100. The method of embodiment II-99, wherein the beta-adrenergic blocker is a timolol.

Embodiment II-101. The method of embodiment II-100, wherein the timolol is timolol maleate or timolol hemihydrate.

Embodiment II-102. The method of any one of embodiments II-99 to II-101, wherein the second drug is a carbonic anhydrase inhibitor.

Embodiment II-103. The method of embodiment II-102, wherein the carbonic anhydrase inhibitor is a dorzolamide or a brinzolamide.

Embodiment II-104. The method of embodiment II-103, wherein the dorzolamide is dorzolamide hydrochloride or dorzolamide base.

Embodiment II-105. The method of any one of embodiments II-99 to II-101, wherein the second drug is a prostaglandin analog or a prostamide analog.

Embodiment II-106. The method of embodiment II-105, wherein the prostaglandin analog is latanoprost, travoprost, tafluprost, or unoprostone, and the prostamide analog is bimatoprost.

Embodiment II-107. The method of embodiment II-105, wherein the second drug is the prostaglandin analog, and the prostaglandin analog is latanoprost.

Embodiment II-108. The method of any one of embodiments II-99 to II-101, wherein the second drug is a rho-kinase inhibitor.

Embodiment II-109. The method of embodiment II-108, wherein the rho-kinase inhibitor is ripasudil or netarsudil.

Embodiment II-110. The method of embodiment II-97 or II-98, wherein the first drug is a prostaglandin analog or a prostamide analog.

Embodiment II-111. The method of embodiment II-110, wherein the prostaglandin analog is latanoprost, travoprost, tafluprost, or unoprostone, and the prostamide analog is bimatoprost.

Embodiment II-112. The method of embodiment II-111, wherein the first drug is the prostaglandin analog, and the prostaglandin analog is latanoprost.

Embodiment II-113. The method of any one of embodiments II-110 to II-112, wherein the second drug is a rho-kinase inhibitor.

Embodiment II-114. The method of embodiment II-113, wherein the rho-kinase inhibitor is ripasudil or netarsudil.

### EXAMPLES

### Example 1: Dry Subconjunctival Delivery of Drug-Eluting Implants Formulated with a Prostaglandin or Cyclosporine

An implantation system loaded with carrier-free drug-eluting microspheres, each of which contains at least one prostaglandin or cyclosporine, is removed from its sterile packaging and placed on a sterile surface. The subject's eye and periocular area are treated with an antiseptic. The eye is then draped in the typical manner for ocular surgery, and an operating microscope and eyelid speculum are properly positioned and placed, respectively. The conjunctiva is grasped with conjunctival forceps.

The implantation system needle is advanced under direct microscopic visualization into the subconjunctival space. The actuator on the device handle is activated (button, slider, lever, and/or wheel). The pusher inside the needle moves distally toward the distal tip of the needle and advances the pre-determined number of microspheres out of the needle into the subconjunctival space. This is visualized with a microscope. Visualization may also be performed with a slit lamp, loupes, or the naked eye. The needle is then withdrawn from the conjunctiva. Antibiotic drops are applied to the eye, and the eyelid speculum is removed.

### Example 2: Dry Delivery of Drug-Eluting Implants Formulated with a Prostaglandin into the Posterior Chamber/Sulcus

An implantation system loaded with carrier-free drug-eluting microspheres, each of which contains at least one prostaglandin, is removed from its sterile packaging and placed on a sterile surface. The subject's eye and periocular area are treated with an antiseptic. The eye is then draped in the typical manner for ocular surgery, and an operating microscope and eyelid speculum are properly positioned and placed, respectively.

The implantation system needle is advanced under direct microscopic visualization through the cornea, near the limbus, into the anterior chamber. The needle tip is advanced to the pupil and just past the pupillary collarette. The needle may be curved or straight. The actuator on the device handle is activated (button, slider, lever, or wheel). The pusher rod inside needle moves forward/distally to tip of needle and moves the pre-determined amount of microspheres out of the needle into the posterior chamber. This is visualized with microscope. Alternatively, prior to implantation of the pre-dosed microspheres, a soft atraumatic catheter is inserted into the posterior chamber. Visualization may also be performed with a slit lamp, loupes, or the naked eye. The needle is then withdrawn from conjunctiva. Antibiotic drops are applied to the eye, and the eyelid speculum is removed.

### Example 3: Dry Subconjunctival Delivery of Drug-Eluting Implants Formulated with a Timolol and a Dorzolamide

An implantation system with a cannula loaded with carrier-free drug-eluting microspheres (e.g. a dry implant formulation), and a handle comprising a housing and an actuator and coupled to the cannula is removed from its sterile packaging and placed on a sterile surface. Optionally, the loaded cannula and the handle are separately packaged, and the cannula is attached to the handle prior to use. A first subset of the pre-loaded microspheres contain a timolol (e.g. timolol maleate or timolol hemihydrate) and a second subset of the pre-loaded microspheres contain a dorzolamide (e.g., dorzolamide hydrochloride, dorzolamide base) or brinzolamide. The first and second subsets of the microspheres may be combined in one implantation unit of a dry implant formulation that comprises both implant subsets. The subject's eye and periocular area are treated with an antiseptic. The eye is then draped in the typical manner for ocular surgery, and an operating microscope (or slit lamp) and eyelid speculum are properly positioned and placed, respectively. The conjunctiva is grasped with conjunctival forceps.

The cannula of the implantation system is advanced under direct microscopic visualization, penetrating the conjunctiva and into a target location in the eye, which may be a subconjunctival space or a sub-Tenon's space. The actuator on the handle is activated (button, slider, lever, and/or wheel). The pusher rod inside the cannula moves distally toward the distal tip of the cannula and releases the pre-loaded microspheres out of the cannula into the target location. The microsphere release is visualized with a microscope or slit lamp. Visualization may also be performed with a slit lamp, loupes, or the naked eye. The cannula is then withdrawn from the conjunctiva. Antibiotic drops are applied to the eye, and the eyelid speculum is removed.

1. A method for treating a condition of an eye of a subject, the method comprising:
implanting a dry implant formulation in the eye of the subject without a carrier, the dry implant formulation comprising a plurality of drug-eluting microparticle implants,
wherein at least one drug is delivered from the plurality of drug-eluting microparticle implants to the eye to reduce a symptom associated with the condition of the eye.

2. The method of claim 1, wherein each of the plurality of drug-eluting microparticle implants comprises a drug-eluting matrix.

3. The method of claim 2, wherein the drug-eluting matrix comprises a bio-erodible polymer and the at least one drug.

4. The method of claim 3, wherein the bio-erodible polymer comprises poly(lactic-co-glycolic acid) (PLGA), poly(lactic acid) (PLA), poly-epsilon-caprolactone (PCA), or poly(glycolic acid) (PGA).

5. (canceled)

6. The method of claim 1, wherein each of the plurality of the drug-eluting microparticle implants has a diameter of between about 1 μm and about 500 μm.

7. (canceled)

8. The method of claim 1, wherein the condition of the eye is glaucoma and/or dry eye disease.

9. The method of claim 1, wherein the dry implant formulation is implanted in one or more of a sulcus, a posterior chamber, an anterior chamber, a vitreous, a sub-Tenon's space, and a subconjunctival space of the eye.

10. The method of claim 1, wherein the at least one drug comprises a prostamide analog or a prostaglandin analog.

11. The method of claim 1, wherein the at least one drug comprises a beta-adrenergic blocker, a carbonic anhydrase inhibitor, a rho-kinase inhibitor, a nerve growth factor, an anti-VEGF antibody, sirolimus, tacrolimus, everolimus, or cyclosporine.

12-13. (canceled)

14. The method of claim 1, wherein the plurality of the microparticle implants in total is configured to deliver the at least one drug to the eye at a rate of between about 1 ng/day and about 50 μg/day.

15. The method of claim 1, wherein the plurality of the microparticle implants in total is configured to deliver the at least one drug to the eye at a rate of between about 5 ng/day and about 2000 ng/day.

16. The method of claim 1, wherein the dry implant formulation is configured to deliver the at least one drug to the eye over a period of time, wherein the period of time is at least 1 month, at least 4 months, at least 6 months, at least 8 months, at least 1 year, at least 2 years, or at least 3 years.

17. The method of claim 1, wherein at least a subset of the plurality of the microparticle implants comprises an imaging agent.

18-20. (canceled)

21. The method of claim 1, wherein the plurality of drug-eluting microparticle implants comprises a first microparticle implant comprising a first drug of the at least one drug and a second microparticle implant comprising a second, different drug of the at least one drug.

22. The method of claim 21, wherein the first drug is a beta-adrenergic blocker.

23. The method of claim 22, wherein the beta-adrenergic blocker is a timolol.

24. (canceled)

25. The method of claim 22, wherein the second drug is a carbonic anhydrase inhibitor.

26. The method of claim 25, wherein the carbonic anhydrase inhibitor is a dorzolamide or a brinzolamide.

27. (canceled)

28. The method of claim 22, wherein the second drug is a prostaglandin analog or a prostamide analog.

29. The method of claim 28, wherein the prostaglandin analog is latanoprost, travoprost, tafluprost, or unoprostone, and the prostamide analog is bimatoprost.

30. (canceled)

31. The method of claim 22, wherein the second drug is a rho-kinase inhibitor. 32-37. (canceled)

38. The method of claim 21, wherein the condition is glaucoma, the first drug is a timolol, and the second drug is a dorzolamide or a brinzolamide, and wherein the dry implant formulation is implanted in sub-Tenon's space.

39. The method of claim 1, wherein each of the plurality of drug-eluting microparticle implants is a microsphere.

40. The method of claim 1, wherein each of the plurality of drug-eluting microparticle implants comprises a bio-erodible polymer, and at least a majority of the plurality of drug-eluting microspheres have been heated above the glass transition temperature of the bio-erodible polymer.

41. The method of claim 1, further comprising a binding agent.

42. The method dry implant formulation of claim 41, wherein the binding agent is selected from the group consisting of: a sugar, a gelatin, a collagen, polyethylene glycol (PEG), a starch, a cellulose, an alginate, and a chitosan.

43. (canceled)

44. The method of claim 1, wherein the dry implant formulation is malleable, and does not readily dissociate into individual implants.

45-114. (canceled)

115. The method of claim 1, wherein the dry implant formulation is a first dry implant formulation and the method further comprises implanting a second dry implant formulation, the second dry implant formulation comprising a second plurality of drug-eluting microparticle implants.

116. The method of claim 115, wherein the first dry implant formulation and the second dry implant formulation are implanted sequentially.

117. The method of claim 115, wherein the second dry implant formulation is implanted in a different location in the eye than the first dry implant formulation.

118. The method of claim 8, wherein the condition of the eye is glaucoma, and the symptom associated with glaucoma is increased ocular pressure.
